(19) United States
(12) Patent Application Publication
(10) Pub. No.: US 2010/0143980 A1
(43) Pub. Date: Jun. 10, 2010

(54) SYNTHESIS OF NOVEL XYLOSIDES AND POTENTIAL USES THEREOF

(76) Inventors: Kuberan Balagurunathan, Salt lake City, UT (US); Ethirajan Manivannan, Williamsville, NY (US); V. Victor Xylophone, Salt Lake City, UT (US); Vy My Tran, Salt Lake City, UT (US); Khiem Nguyen, Salt Lake City, UT (US)

Correspondence Address: Workman Nydegger;1000 Eagle Gate Tower, 60 East South Temple, Salt Lake City, UT, 84111, US

(21) Appl. No.: 12/528,172

(22) PCT Filed: Feb. 15, 2008
(86) PCT No.: PCT/US08/54176

§ 371 Date: Jan. 27, 2010

Related U.S. Application Data
(60) Provisional application No. 60/891,168, filed on Feb. 22, 2007.
(60) Provisional application No. 60/896,230, filed on Mar. 21, 2007.

(51) Int. Cl. C12P 19/04; C07H 19/056; C07H 19/06

(52) U.S. Cl. 435/101; 536/28.7; 536/28.53

(57) ABSTRACT

The present invention includes a xyloside for use in inducing synthesis of a glycosaminoglycan in a cell, the xyloside having a chemical structure of one of Formula (1), Formula (2), Formula (3), Formula (4), Formula (5), Formula (6), Formula (7), Formula (8), Formula (9), or Formula (10) as shown herein. Also, the present invention includes a method of making a xyloside for use in inducing synthesis of a glycosaminoglycan in a cell, wherein the method is performed with “Click” chemistry. Additionally, the present invention includes a method of administering a xyloside so as to induce synthesis of a glycosaminoglycan in a cell.

This application claims the benefit of U.S. Provisional Patent Application No. 60/891,168 filed on Feb. 22, 2007, and U.S. Provisional Patent Application No. 60/896,230 filed on Mar. 21, 2007; both of which are incorporated herein by reference.

### BACKGROUND OF THE INVENTION

1. The Field of the Invention

The present invention relates to novel xylosides that induce/prime the synthesis of glycosaminoglycans (GAGs) in cells.

2. The Relevant Technology

Proteoglycans are composed of a core protein and several complex glycosaminoglycan (GAG) polysaccharide side chains. Heparan Sulfate (HS), Chondroitin Sulfate (CS) and Dermatan Sulfate (DS) belong to the family of GAGs. In humans, these GAG side chains are shown to regulate many biological functions including wound healing, cell signalling, cell differentiation, angiogenesis, blood clotting, tumor cell migration. GAGs consist of repeating disaccharide units of hexosamine and uronic acid, and are covalently attached to a serine residue of the core protein via a specific linkage tetrasaccharide (FIG. 1A). The very first step in GAG synthesis is xylosylation of a serine residue of the core protein, followed by assembly of a tetrasaccharide unit that serves as an acceptor for elongation of GAG chains (FIG. 1B).

Therefore, it is desirable to have improved reagents and methods of promoting/inducing the biosynthesis of GAGs.

### BRIEF SUMMARY OF THE INVENTION

In one embodiment, the present invention includes a xyloside for use in inducing synthesis of a glycosaminoglycan in a cell, the xyloside having a chemical structure of one of Formula 1, Formula 2, Formula 3, Formula 4, Formula 5, Formula 6, Formula 7, Formula 8, Formula 9, or Formula 10 as shown herein.

In one embodiment, the xyloside has a chemical structure of one of Formula 1, Formula 2, Formula 3, or Formula 4. In this embodiment, n is from 0 to 10; m is from 0 to 10; X is one of S, O, N, or C; and R-R″″′ are each independently one of H, CH2OH, halogen, COOH, acetyl-oxy, monosaccharide, disaccharide, oligosaccharide, of OH.

In one embodiment, the xyloside has a chemical structure of one of Formula 5 or Formula 6. In another embodiment, the xyloside has a chemical structure of one of Formula 7 or Formula 8. In another embodiment, the xyloside has a chemical structure of one of Formula 9 or Formula 10. The R groups are as described herein.

The R groups of the xyloside can be on the sugar and/or agylcon groups and/or can be the agylcon group. The R (e.g., R, R*, R′-R″″′, and/or R1-R11) groups are each individually selected from H, OH, CH2OH, halogen, F, Cl, Br, I, alkoxy, methoxy, NO2, unsubstituted aliphatic group, a substituted aliphatic group, a halo substituted aliphatic group, a straight chain aliphatic group, a branched chain aliphatic group, a cyclic aliphatic group, an aliphatic group having at least one hetero chain atom, a cyclic aliphatic group having at least one hetero ring atom, an unsubstituted aromatic group, a substituted aromatic group, a halo substituted aromatic group, a polyaromatic group, a substituted polyaromatic group, an aromatic group having at least one hetero ring atom, a polyaromatic group having at least one hetero ring atom, a nucleoside, a nucleotide, a carbohydrate, monosaccharide, disaccharide, oligosaccharide, a polysaccharide; an amino acid, a polypeptide, adenine, guanine, thymine, uracil, cytosine, combinations thereof, and derivatives thereof. Thus, the R groups can all be the same or all different, as is possible. Specific examples include H, CH2OH, halogen, COOH, acetyl-oxy, monosaccharide, disaccharide, oligosaccharide, or OH.

The xylosides of the present invention can include R* (i.e., agylcon groups) can be H, OH, or CH2OH, or at least one of the groups shown in Tables 1-5, derivative thereof, or combination thereof. The R group (e.g., R, R′-R″″′, and/or R1-R11) an any R* group can be as described herein.

In one embodiment, a xyloside in accordance with the present invention can be characterized by at least one of the following: when R′ is H, then R″ is OH; when R′ is OH or F, then R″ is H; when R′″ is OH, then R″″ is H; when R′″ is H, then R″″ is OH; at least one of R, R′, R″, R″′, or R″″ is H; at least one of R, R′, R″, R′″, or R″″ is OH; at least one of R, R′, R″, R″′, or R″″ is F; at least one of R is H, R′ is OH, R″ is H, R′″ is OH, or R″″′ is H; the aliphatic group of R* has a main chain of 1 to 10 carbons; or the aromatic group or polyaromatic group of R* has from 1 to 3 aromatic rings.

In one embodiment, the m and/or n of a xyloside as shown in Formulas 1-10 are each independently from about 0 to about 10, more preferably from about 0 to about 5, and most preferably m is 0 or 1 and/or n is 0 or 1.

In one embodiment, the X of a xyloside as shown in Formulas 1-10 is one of a bond, S, SO2, O, N, or C.

In one embodiment, the present invention includes a method of making a xyloside for use in inducing synthesis of a glycosaminoglycan in a cell. Such a method includes obtaining an N3-containing reagent having a chemical structure as in one of Reagent 1A, Reagent 1B, Reagent 1C, or Reagent 1D as shown herein, wherein at least one of R, R′, R″, R″′, R″″, or R″″′, as shown herein, is protected by a protecting group that is capable of being deprotected without degrading the xyloside; obtaining an alkynyl-containing reagent having a chemical structure as in one of Reagent 2A, Reagent 2B, or Reagent 2C as shown herein; reacting one of Reagents 1A-1D with one of Reagents 2A-2C; and deprotecting the at least one of R, R′, R″, R′, R″″, or R″″′ that is protected by the protecting group so as to arrive at the at least one of R, R′, R″, R′, R″″, or R′″″ so as to obtain the xyloside.

In another embodiment, the present invention includes another method of making a xyloside for use in inducing synthesis of a glycosaminoglycan in a cell. Such a method can include: obtaining an alkynyl-containing reagent having a chemical structure as in one of Reagent 3A or Reagent 3B as shown herein, wherein at least one of R, R′, R″, R′″, R″″, or R″″′, as shown herein, is protected by a protecting group that can be deprotected without degrading the xyloside; obtaining an N3-containing reagent having a chemical structure as in Reagent 4A as shown herein; reacting one of Reagent 3A-3B with Reagent 4A; and deprotecting the at least one of R, R′, R″, R″′, R″″, or R′″″ that is protected by the protecting group so as to arrive at the at least one of R, R′, R″, R″′, R″″, or R″″′ so as to obtain the xyloside.

In one embodiment, the present invention includes a method of using a xyloside for inducing synthesis of a glycosaminoglycan in a cell. Such a method can include providing a xyloside; introducing the xyloside into the cell; and maintaining the cell under conditions in which the xyloside is capable of inducing the cell to synthesize the glycosaminoglycan. The xyloside can be as described herein. For example, the xyloside can have a chemical structure of one of Formula 1, Formula 2, Formula 3, Formula 4, Formula 5, Formula 6, Formula 7, Formula 8, Formula 9, or Formula 10 as shown herein.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1A illustrates a schematic representation of a proteoglycan linkage tetrasaccharide sequence.

FIG. 1B illustrates that xylosides of the present invention initiate glycosaminoglycan biosynthesis in mammalian cells.

FIG. 2 illustrates a graph showing a comparison of priming activity of various xylosides of the present invention.

FIG. 3 illustrates a graph showing the priming ability of various xylosides of the present invention.

FIG. 5A illustrates a chemical schematic representation of a HS GAG chain.

FIG. 5B illustrates a chemical schematic representation of the basic disaccharide structures of HS, CS and DS.

FIG. 6 illustrates generalized synthesis procedure for prepared xylosides in accordance with the present invention.

FIGS. 7A-7G illustrate schematic representations of chemical synthesis protocols for preparing the reagents and xylosides of the present invention through “Click” Chemistry techniques.

FIG. 8A illustrates a schematic representation of an exemplary chemical synthesis protocol for preparing the S-xylosides of the present invention.

FIG. 8B illustrates a schematic representation of an exemplary chemical synthesis protocol for preparing the C-xylosides of the present invention.

FIG. 9 illustrates chemical structures of exemplary xylosides of the present invention.

FIG. 10A-10AI are graphs of HPLC analysis of corresponding xylosides in accordance with the present invention.

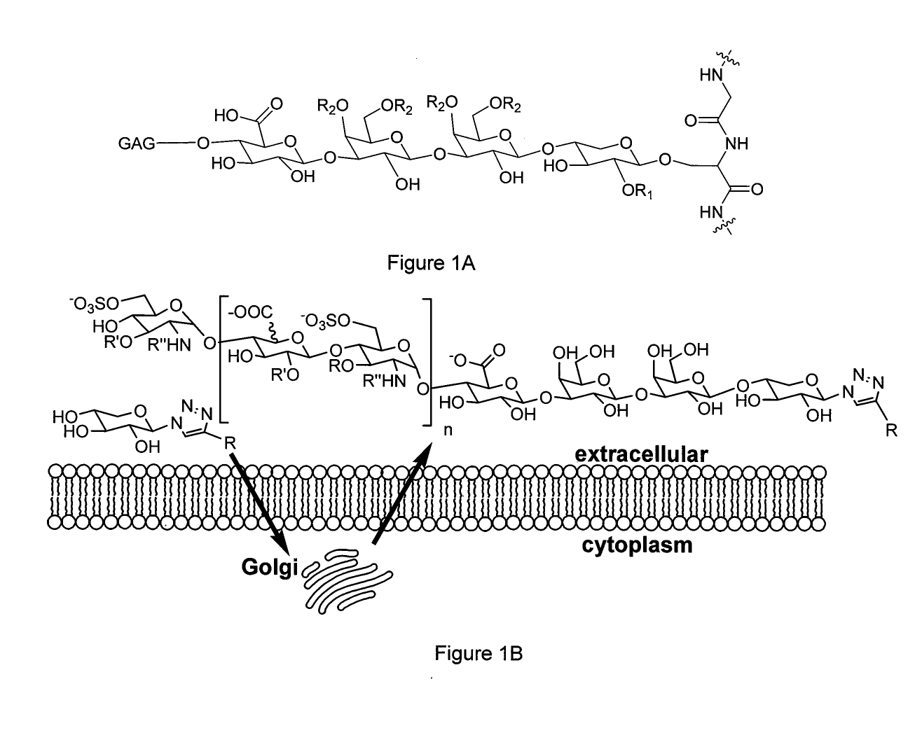

### DETAILED DESCRIPTION OF THE PREFERRED EMBODIMENTS

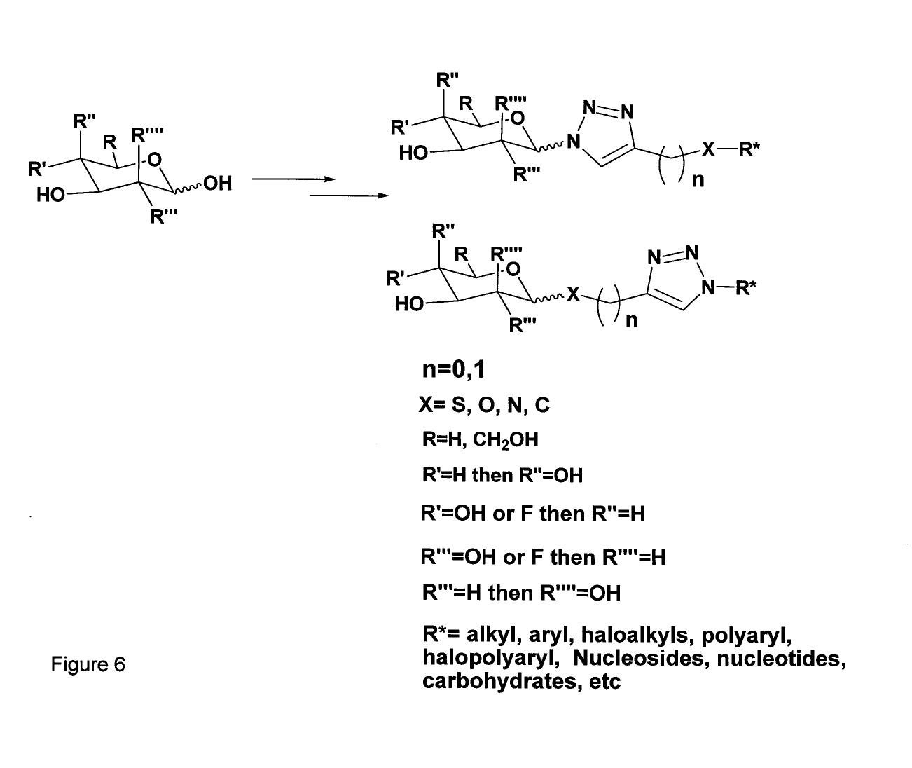

Glycosaminoglycans (GAGs) play a vital role in various physiological functions such as wound healing, cell signaling, endothelial proliferation, angiogenesis, blood clotting, prevention of infection, fertilization, and the like. Despite their important roles, the factors that regulate their synthesis and their biosynthetic mechanisms are poorly understood. To unravel the mechanism of GAG biosynthesis and also to investigate the structural and priming activity relationship, we have synthesized a variety of novel xylosides with various aglycone moieties by using 1,3-dipolar addition reaction (“Click” chemistry) and/or other chemical synthesis methods that can be adapted to synthesize the xylosides of the present invention. By performing the click chemistry on the suitable precursors, we have synthesized a library of xylosides having various glycosidic linkages such as O, N, C, S, and SO2, in a rapid manner (FIG. 6). The synthesized compounds were examined for their ability to induce heparan sulfate (HS), chondroitin sulfate (CS) and/or Dermatan sulfate (DS) in CHO cells and also in micro and macro endothelial cells. Further, the induced HS or DS polysaccharides were isolated and evaluated for their potential priming activity.

GAG chains can also be synthesized in the cells by using simple O-aryl xylosides without a core protein. Thus, xylosides with hydrophobic aglycone can compete with endogenous core protein acceptor sites for the assembly of GAG chains in Golgi. It is observed that the quantity and composition of these GAG units entirely depends on the structure of the aglycone moiety. In addition, S- and C-xylosides, which are more stable than O-xylosides, are also shown to prime GAG chains but only a select few of these xylosides were examined in detail. A C-xyloside has been synthesized by an elegant approach, but this molecule was surprisingly unable to prime a detectable amount of GAGs in fibroblast cells. Despite the fact that O-xylosides are metabolically less stable than the S- and C-xylosides, the less stable O-xylosides can be used in model organisms and demonstrated the role of GAGs in developmental biology.

It is surprising to note from our results that by replacing oxygen atom with carbon, nitrogen, or sulfur atoms, the xylosides showed better activity in some cases and also selectively synthesized HS, CS, or DS polysaccharides. We also observed that some structural variations on aglycone moiety are forbidden while others are permissive for priming GAG chains.

The xylosides of the present invention can be used in studies on the induced GAG chains, and may shine a light on the biosynthesis of proteoglycans, their sulfation pattern, and hence their mechanism of biosynthesis. Also, the xylosides of the present invention can be used as potential drugs for blood clotting, wound healing and in general the prevention of cardiovascular diseases.

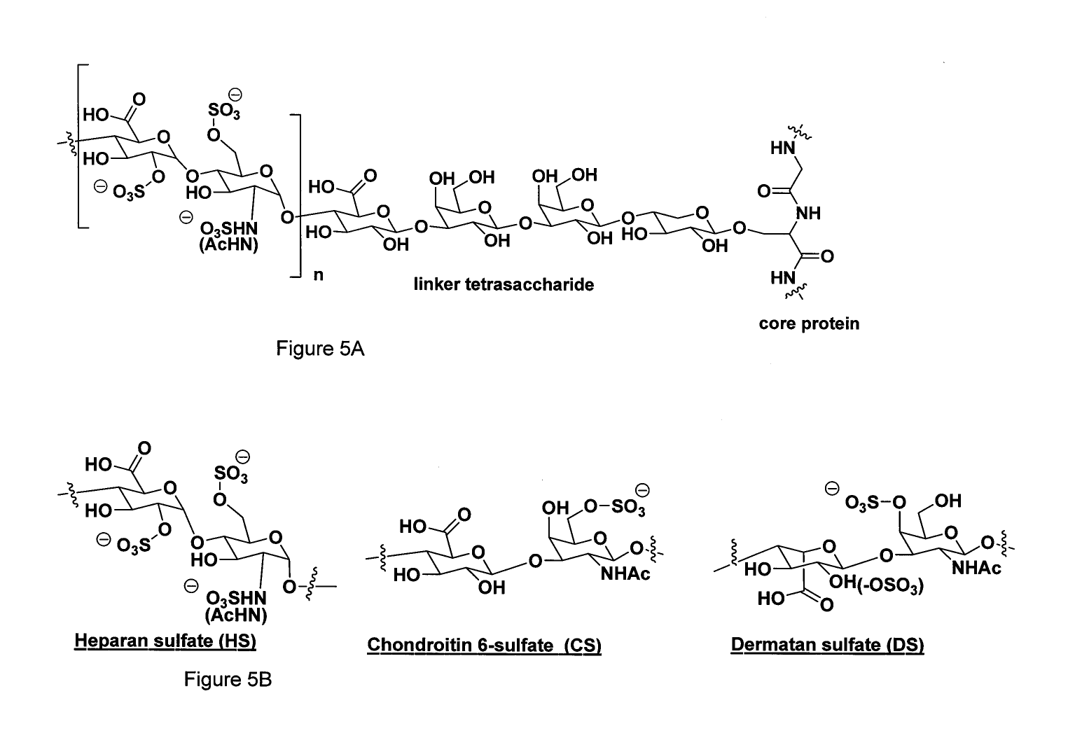

Proteoglycans are composed of a protein moiety and a complex polysaccharide moiety which is responsible for many biological activities in our body such as wound healing, cell signaling, blood clotting, endothelial proliferation, angiogenesis, and the like. HS is one such polysaccharide, consists of repeating disaccharides (FIG. 5A) units which are covalently attached to a serine residue of the core protein via a specific tetrasaccharide linkers. The first step in GAG synthesis is xylosylation of a serine residue of the core protein. A specific linker tetrasaccharide is assembled and serves as an acceptor for elongation of GAG chains. It is still obscure which factor is responsible for synthesis of HS, CS, or DS disaccharide (FIG. 5B) repeating units.

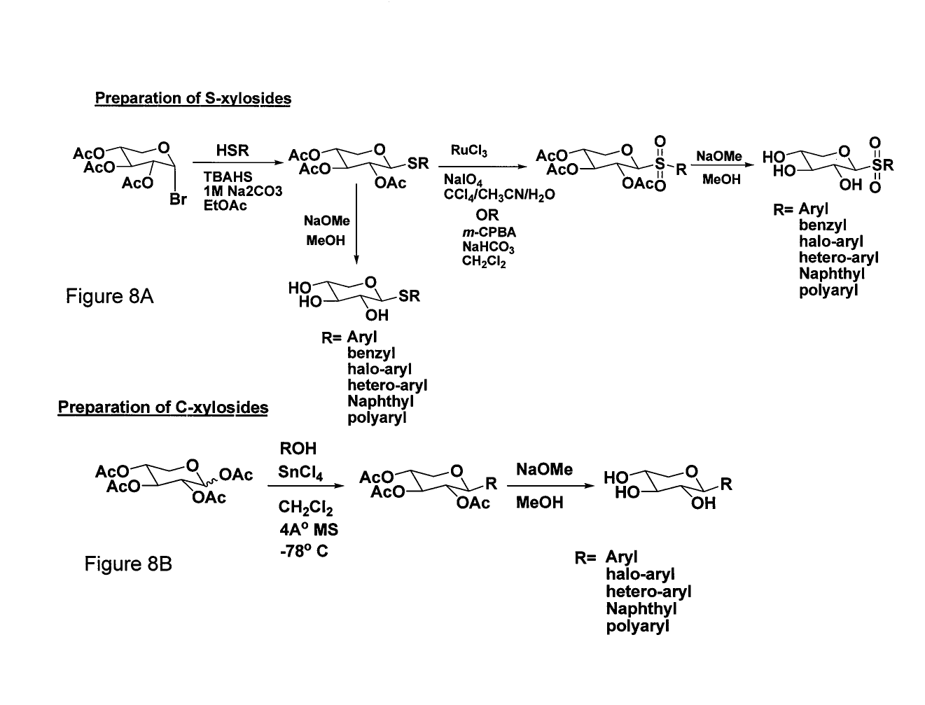

Over the last decade, C-glycosides have been the subject of considerable interest in carbohydrates, enzymatic, and metabolic chemistry as it is not cleavable by hydrolytic enzymes. Because of its stability against hydrolysis when compared to O-glycosides, it can penetrate through the membranes without getting cleaved, and hence it is very stable under physiological conditions. Thus, the half life of C-glycosides is increased and also its bioavailability. GAG chains can also be synthesized in the cells by using phenylxylosides without the core protein. Thus, xylosides with hydrophobic aglycone can compete with endogenous core protein acceptor sites for the assembly of GAG chains in Golgi. It is observed that the quantity and composition of these GAG units entirely depends on the structure of the aglycone which may reflect the selective partitioning of primers into different branches of biosynthetic pathways. Though S-xylosides, which can be synthesized as shown in FIG. 8A, and C-xylosides, which can be synthesized as shown in FIG. 8B, are more stable than O-xylosides. Such S-xylosides and C-xylosides of the present invention are shown to prime GAG chains. The S-xylosides and C-xylosides can include the R group being selected from the group consisting of H, OH, CH2OH, an unsubstituted aliphatic group, a substituted aliphatic group, a halo substituted aliphatic group, a straight chain aliphatic group, a branched chain aliphatic group, a cyclic aliphatic group, an aliphatic group having at least one hetero chain atom, a cyclic aliphatic group having at least one hetero ring atom, an unsubstituted aromatic group, a substituted aromatic group, a halo substituted aromatic group, a polyaromatic group, a substituted polyaromatic group, an aromatic group having at least one hetero ring atom, a polyaromatic group having at least one hetero ring atom, a nucleoside, a nucleotide, a carbohydrate, monosaccharide, disaccharide, oligosaccharide, a polysaccharide; an amino acid, a polypeptide, combinations thereof, and derivatives thereof. Additionally, the OH groups can be the R, R′, R″, R″′, R″″, and/or R″″′ as described herein.

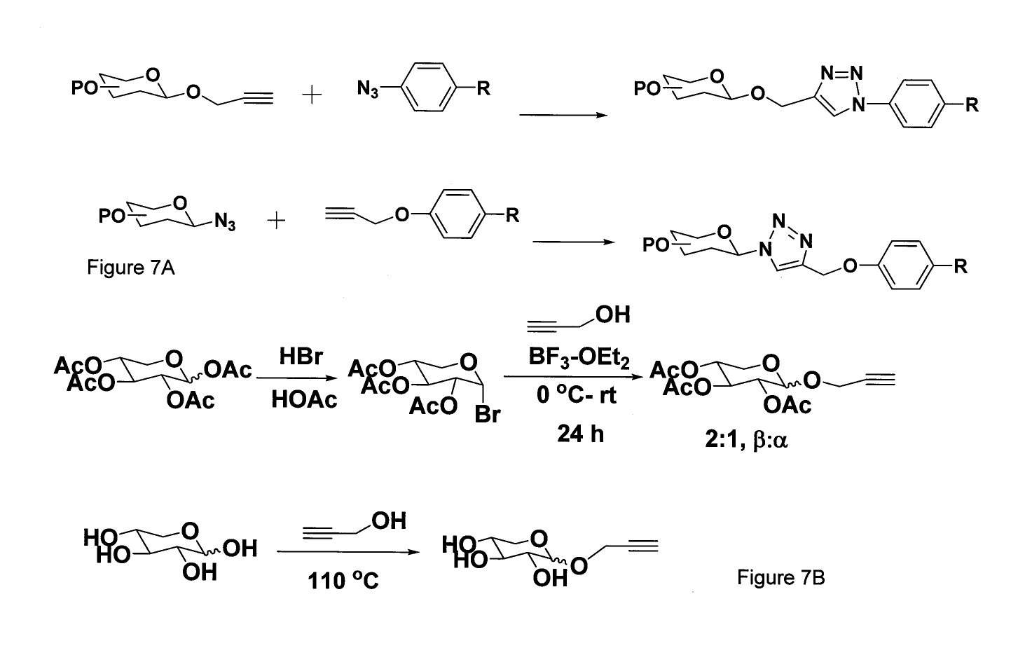

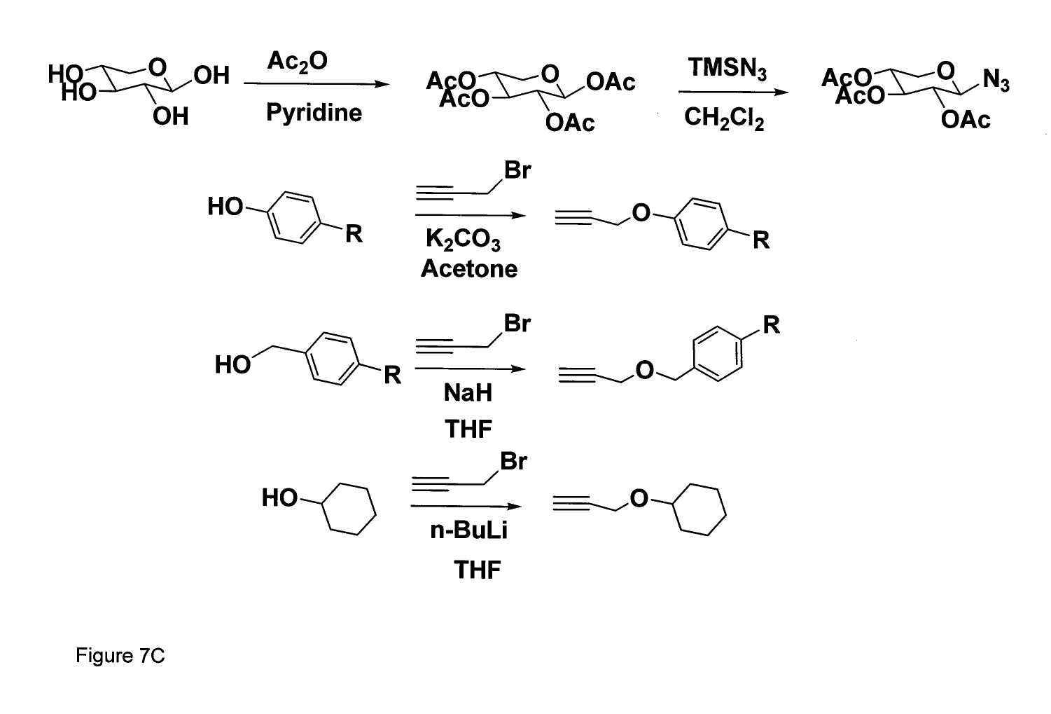

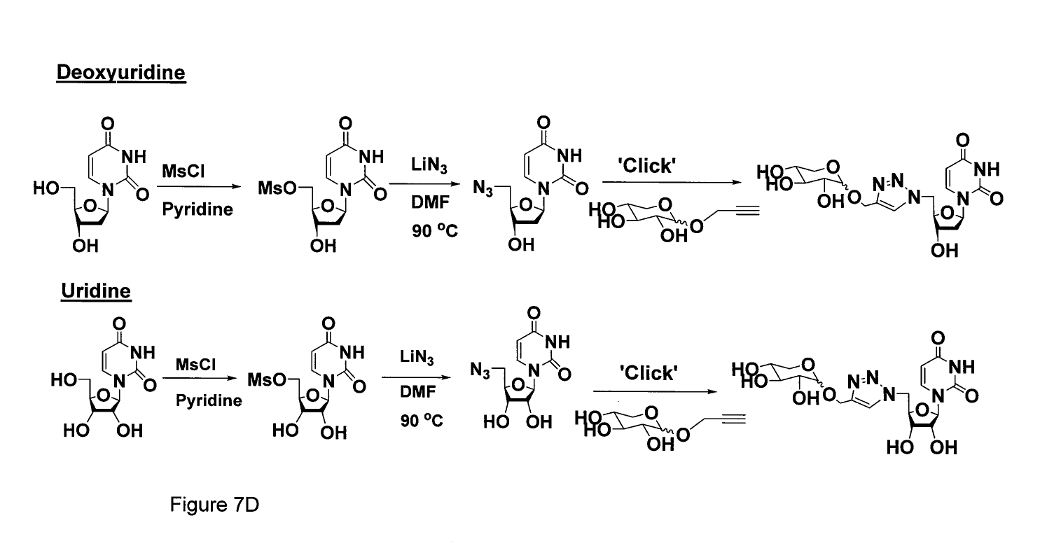

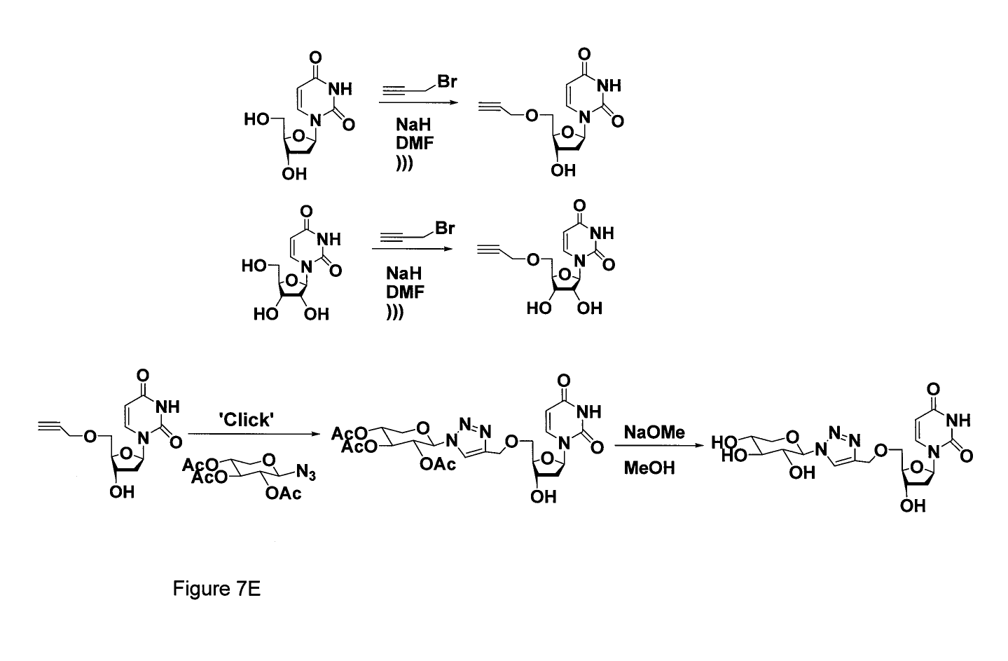

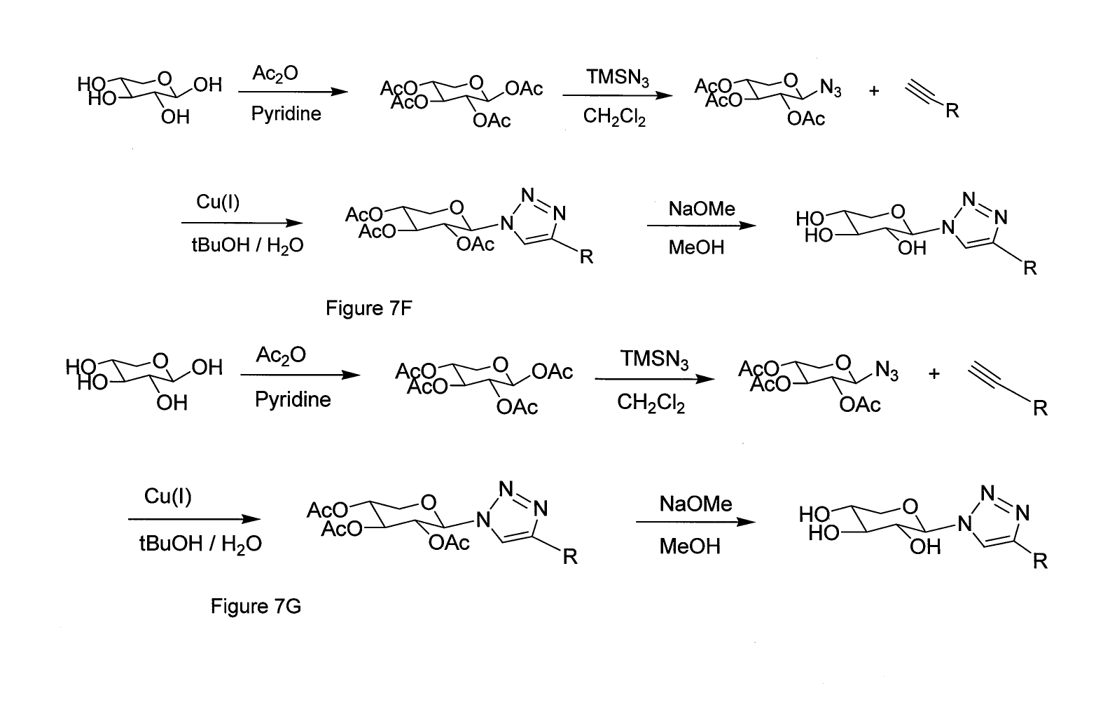

In one embodiment, the present invention includes a xyloside and method for producing the xyloside, which is obtained by synthesizing a xyloside which has a triazole moiety in between the aglycone and the sugar unit with different chain lengths. In this regard, a library of triazole xylosides as described herein can be synthesized using click chemistry as shown in FIG. 7A-7F (Click Chemistry). The preparation of reagents for use in Click Chemistry is shown in FIG. 7G, which can be applied to substantially all reagents for preparing the xylosides as described herein. This approach introduces diverse set of aglycones rather quickly and allows one to examine the effect of aglycone moieties on the stimulation of GAGs in a rigorous manner. Furthermore, triazole xylosides are predicted to be metabolically more stable and could lead to long lasting biological activity, and thus “click” xylosides can be more advantageous to study their effect during development in model organisms. In this regard, we have synthesized various xylosides (Tables 1-5; Formulas A-AD) and characterized their ability to stimulate the production of glycosaminoglycans.

The absence of GAG synthesis on endogenous proteoglycans in mutant pgsA-745 makes these cells a convenient system for studying priming of GAGs by exogenous xylosides, such as the xylosides of the present invention. At the outset, a series of alpha and beta-D-xylosides were fed to a mutant CHO cell line that lacks xylosyltransferase (pgsA-745). All of alpha-D-xylosides prime fewer GAGs compared to their beta-xyloside counterparts. Previous studies suggest that alpha-D-xylosides can not prime glycosaminoglycan production, and in fact early experiments with beta-xylosides often used alpha-xyloside as a negative control. The beta-D-thiophenyl xyloside has been used as a positive control since previous studies demonstrated its GAG priming ability.

Simple triazole linked beta-xylosides prime slightly better than the control compound. When a phenyl ring is attached to a triazole moiety, it primed nearly 8 times more GAG chains compared to the control compound and a simple triazole xyloside. This demonstrated the importance of hydrophobic group, which confers priming activity to the xyloside.

Heteroatom substitutions in the phenyl rings were examined to determine whether or not it would differentially modulate GAG priming. Pyridyl and phenyl rings attached to the triazole moiety were also compared. Pyridyl substituted xylosides primed less efficiently than phenyl substituted xyloside derivatives, which is in accordance with an earlier observation. To test the effect of a phenyl ring substitution at the para position on the priming ability, a set of xylosides were synthesized with the following substitutions: F, Cl, Br, I, —NO2, and OCH3. Experimental data suggest that any substitution at a para position of phenyl ring dramatically reduces the priming ability of xylosides except the —OCH3 group. The priming ability gradually decreases with increasing atomic weight of halogen atoms. Methoxyl groups retain priming ability while NO2 substitution dramatically decreases the priming ability by 12-fold.

The effect of biphenyl and napthyl rings on the priming ability of xylosides was also analyzed. In general, the napthyl moiety is more conducive for priming activity than the biphenyl moiety. Nevertheless, addition of a halogen atom, such as Br, to either a napthyl or a biphenyl moiety decreased the priming activity of xylosides. On the other hand, saturation of one of the napthyl aromatic rings did not significantly influence printing activity.

Phenanthrene, which is more hydrophobic than napthalene, was predicted to confer more priming activity. It was surprising to note that such a polyaromatic ring did not have substantial activity in priming GAG chains, and was found to be cytotoxic to the cells when they are exposed to such a primer for a long period of time. Overall, xylosides having a single phenyl ring were found to be better than a biphenyl or a polyaromatic moiety for priming.

The spacing of a phenyl ring was examined. Removal of an oxygen spacer decreased the biological activity. When a methylene group was introduced between the oxygen and the phenyl ring (benzyl), priming ability was retained. Introduction of a CH2OH group on the benzyl ring at the meta position significantly increased the priming ability. This indicates that hydrogen bonding interactions between the xyloside and the GAG biosynthetic machinery may provide for optimal priming.

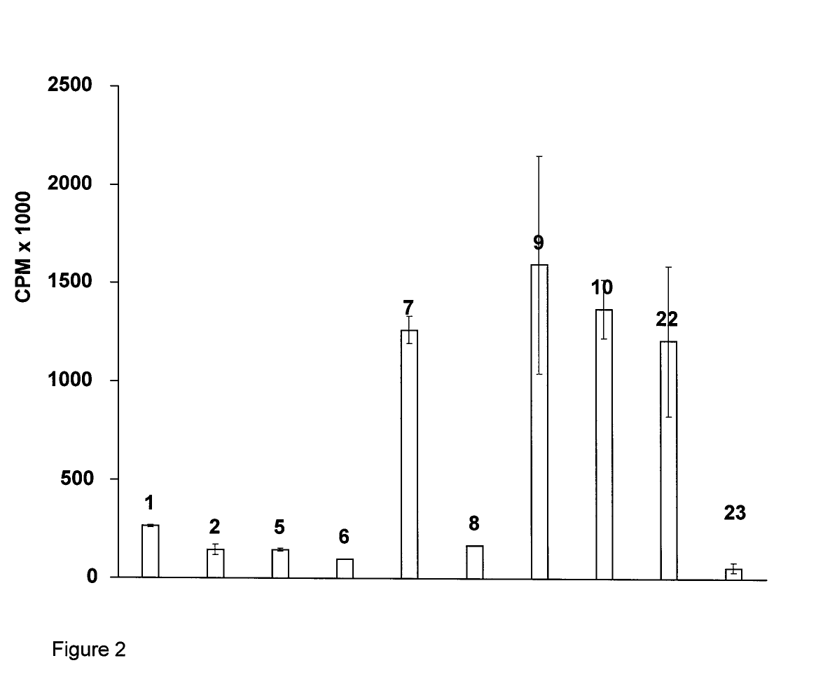

Xylosides having naphthyl rings as an aglycone are shown to produce more HS chains than CS/DS chains. We extensively examined the effect of different aglycone moieties on their ability to prime HS, CS and/or DS and the results are summarized in FIGS. 2-4.

In accordance with the present invention, a library of xylosides, such as S—, C—, and/or triazole xylosides with extensive aglycone variations can be synthesized, tested, and utilized for their GAG priming activity. Certain modifications are found to be more permissive for the stimulation of GAG biosynthesis. These molecules predictably have a longer in vivo half-life, which is likely to influence the biological actions of glycosaminoglycans at much greater level in animal models. The stimulated synthesis of core protein free glycosaminoglycan side chains compete with the endogenous proteoglycans for binding to protein ligands at the cell surface, and thereby are capable of modulating cell behavior. The primed GAG chains can also be screened for their biological activity and for their role in cardiovascular/developmental biology.

In one embodiment, metabolically stable xylosides for use in vivo require much lower dosages to perform the complex biological roles, such as in embryonic systems which are known to undergo numerous morphological changes in a spatiotemporal manner.

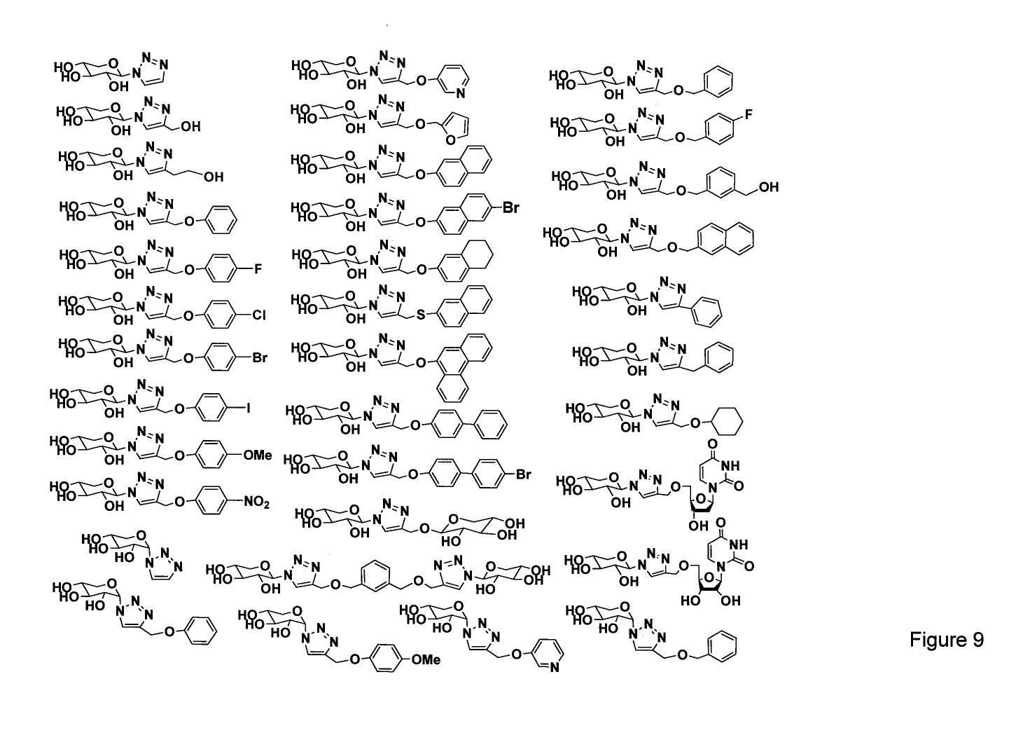

FIG. 9 provides chemical formulas for exemplary xylosides in accordance with the present invention. However, it should be understood that the OH groups and agylcone groups can be modified, dervatized, substituted, and combined under the scope of the present invention. It should be understood that a derivative of a xyloside of the present invention has an atom or substituent substituted for an atom or substituent shown on the chemical formulas depicted herein. Such dervatization to provide a library of compounds is well known in the art of combinatorial chemistry.

In one embodiment, the present invention includes a xyloside for use in inducing synthesis of a glycosaminoglycan in a cell, the xyloside having a chemical structure of one of Formula 1, Formula 2, Formula 3, Formula 4, Formula 5, Formula 6, Formula 7, Formula 8, Formula 9, or Formula 10 as follows.

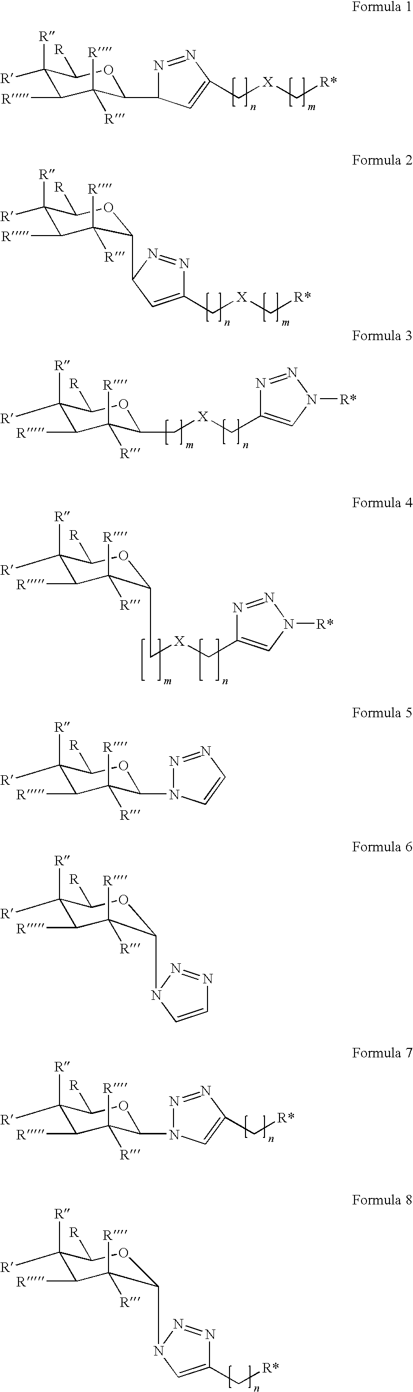

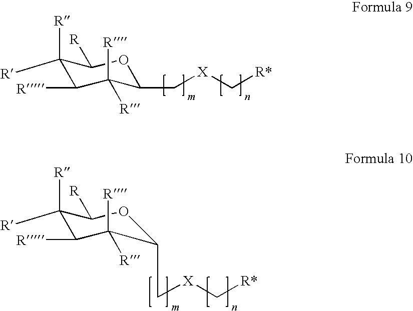

Formulas 1-10 are characterized by the following: n is from 0 to 10; m is from 0 to 10; X is one of a bond, S, SO2, O, N, or C; R, R′, R″, R″′, R″″, and/or R″″′ are each independently one of H, CH2OH, halogen, COOH, acetyl-oxy, monosaccharide, disaccharide, oligosaccharide, or OH; and R* is selected from the group consisting of H, OH, CH2OH, an unsubstituted aliphatic group, a substituted aliphatic group, a halo substituted aliphatic group, alkoxy, a straight chain aliphatic group, a branched chain aliphatic group, a cyclic aliphatic group, an aliphatic group having at least one hetero chain atom, a cyclic aliphatic group having at least one hetero ring atom, an unsubstituted aromatic group, a substituted aromatic group, a halo substituted aromatic group, a polyaromatic group, a substituted polyaromatic group, an aromatic group having at least one hetero ring atom, a polyaromatic group having at least one hetero ring atom, a nucleoside, a nucleotide, a carbohydrate, monosaccharide, disaccharide, oligosaccharide, a polysaccharide; an amino acid, a polypeptide, combinations thereof, and derivatives thereof.

In one embodiment, the xyloside has a chemical structure of one of Formula 1, Formula 2, Formula 3, or Formula 4. In this embodiment, n is from 0 to 10; m is from 0 to 10; X is one of S, O, N, or C; and R-R″″′ are each independently one of H, CH2OH, halogen, COOH, acetyl-oxy, monosaccharide, disaccharide, oligosaccharide, or OH.

In one embodiment, the xyloside has a chemical structure of one of Formula 5 or Formula 6. In another embodiment, the xyloside has a chemical structure of one of Formula 7 or Formula 8. In another embodiment, the xyloside has a chemical structure of one of Formula 9 or Formula 10. The R groups are as described herein.

It should be understood that standard chemistry terminology and symbols are used herein with respect to the xylosides, substituents, R groups, m, n, X, scaffolds, agylcone groups, sugar groups, and the like. As such, the it should be understood that the substituents, R groups, scaffolds, agylcon groups, xylosides, substituents, R groups, scaffolds, agylcone groups, sugar groups, and the like can be combined to be formed into a novel xyloside as described herein by any atom or bond therebetween as possible. Also, it should be understood that the xylosides, substituents, scaffolds, agylcone groups, sugar groups, and the like that have R groups can include one or more R groups as described.

Moreover, aliphatic and aromatic rings that are shown to with a bond originating from the center of the ring and being directed to R1-Rn should be understood that the ring can have from 1 to n substituents on the ring, wherein n indicates the number of total possible substituents. For example, a 6-membered aryl group can have R1-R5 substituents, which can be the same substituent or different, on any of the possible substituent sites. In another example, a 6-membered cycloalkyl can have R1-R11 substituents, which can be the same substituent or different, on any of the possible substituent sites.

The R groups can be on the sugar and/or agylcon groups and/or can be the agylcon group. The R (e.g., R, R*, R′-R″″′, and/or R1-R11) groups are each individually selected from H, OH, CH2OH, halogen, F, Cl, Br, I, alkoxy, methoxy, NO2, unsubstituted aliphatic group, a substituted aliphatic group, a halo substituted aliphatic group, a straight chain aliphatic group, a branched chain aliphatic group, a cyclic aliphatic group, an aliphatic group having at least one hetero chain atom, a cyclic aliphatic group having at least one hetero ring atom, an unsubstituted aromatic group, a substituted aromatic group, a halo substituted aromatic group, a polyaromatic group, a substituted polyaromatic group, an aromatic group having at least one hetero ring atom, a polyaromatic group having at least one hetero ring atom, a nucleoside, a nucleotide, a carbohydrate, monosaccharide, disaccharide, oligosaccharide, a polysaccharide; an amino acid, a polypeptide, adenine, guanine, thymine, uracil, cytosine, combinations thereof, and derivatives thereof. Thus, the R groups can all be the same or all different, as is possible. Specific examples include H, CH2OH, halogen, COOH, acetyl-oxy, monosaccharide, disaccharide, oligosaccharide, or OH.

The xylosides of the present invention can include R* (i.e., agylcon groups) can be H, OH, or CH2OH, or at least one of the groups shown in Tables 1-5, derivative thereof, or combination thereof. The R group (e.g., R, R′-R″″′, and/or R1-R11) an any R* group can be as described herein.

In one embodiment, a xyloside in accordance with the present invention can be characterized by at least one of the following: when R′ is H, then R″ is OH; when R′ is OH or F, then R″ is H; when R″′ is OH, then R″″ is H; when R″′ is H, then R″″ is OH; at least one of R, R′, R″, R″′, or R″″ is H; at least one of R, R′, R″, R′″, or R″″ is OH; at least one of R, R′, R″, R″′, or R″″ is F; at least one of R is H, R′ is OH, R″ is H, R′″ is OH, or R″″ is H; the aliphatic group of R* has a main chain of 1 to 10 carbons; or the aromatic group or polyaromatic group of R* has from 1 to 3 aromatic rings.

In one embodiment, the m and/or n of a xyloside as shown in Formulas 1-10 are each independently from about 0 to about 10, more preferably from about 0 to about 5, and most preferably m is 0 or 1 and/or n is 0 or 1.

In one embodiment, the X of a xyloside as shown in Formulas 1-10 is one of a bond, S, SO2, O, N, or C.

In one embodiment, the present invention includes a method of making a xyloside for use in inducing synthesis of a glycosaminoglycan in a cell. Such a method includes obtaining an N3-containing reagent having a chemical structure as in one of Reagent 1A, Reagent 1B, Reagent 1C, or Reagent 1D, wherein at least one of R, R′, R″, R″′, R″″, or R′″″ is protected by a protecting group that is capable of being deprotected without degrading the xyloside; obtaining an alkynyl-containing reagent having a chemical structure as in one of Reagent 2A, Reagent 2B, or Reagent 2C; reacting one of Reagents 1A-1D with one of Reagents 2A-2C; and deprotecting the at least one of R, R′, R″, R″′, R″″, or R′″″ that is protected by the protecting group so as to arrive at the at least one of R, R′, R″, R″′, R″″, or R″″′ so as to obtain the xyloside.

In another embodiment, the present invention includes another method of making a xyloside for use in inducing synthesis of a glycosaminoglycan in a cell. Such a method can include: obtaining an alkynyl-containing reagent having a chemical structure as in one of Reagent 3A or Reagent 3B, wherein at least one of R, R′, R″, R″′, R″″, or R″″′ is protected by a protecting group that can be deprotected without degrading the xyloside; obtaining an N3-containing reagent having a chemical structure as in Reagent 4A; reacting one of Reagent 3A-3B with Reagent 4A; and deprotecting the at least one of R, R′, R″, R″′, R″″, or R″″′ that is protected by the protecting group so as to arrive at the at least one of R, R′, R″, R″′, R″″, or R″″′ so as to obtain the xyloside.

The reagents and R groups are as described herein.

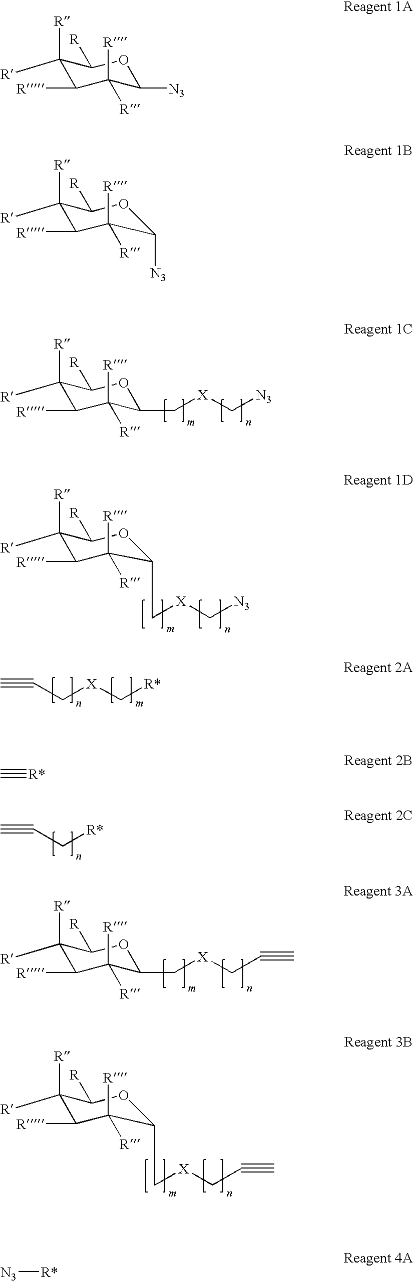

In one embodiment, the present invention includes a method of using a xyloside for inducing synthesis of a glycosaminoglycan in a cell. Such a method can include providing a xyloside; introducing the xyloside into the cell; and maintaining the cell under conditions in which the xyloside is capable of inducing the cell to synthesize the glycosaminoglycan. The xyloside can be as described herein. For example, the xyloside can have a chemical structure of one of Formula 1, Formula 2, Formula 3, Formula 4, Formula 5, Formula 6, Formula 7, Formula 8, Formula 9, or Formula 10.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references recited herein are incorporated herein by specific reference in their entirety.

### EXAMPLES

### Example 1

### Procedure for Synthesizing “Click” Xylosides

All moisture-sensitive and air-sensitive reactions were performed under a nitrogen atmosphere using oven dried glass wares. Anhydrous solvents were purchased and used or dried over standard drying agents and freshly distilled prior to use. Commercially available molecular sieves were used, after activation. Reactions were monitored by TLC on silica gel 60 FZ54 with detection by sulfuric acid, KMnO4 indicator or by Von's reagent. Flash chromatography columns were prepared by hand using silica gel 60 (230-400 mesh) and were 1 Un under pressures of 5-7 psi.

All synthetic compounds were characterized by 1H and 13C-NMR spectroscopic analysis. The spectroscopically characterized compounds were confirmed for their final structures and for the molecular weights using high resolution mass spectrometers (MALDI or ESI-TOF).

To a solution of alkyne (1 mmol) and azide (1 mmol) in t-butanol and water (1:1) solvent mixture were added sodium ascorbate (0.1 mmol) followed by Cu2SO4 5H2O (0.01 mmol) at room temperature, and the mixture was stirred for 12 hours (specific time or disappearance of one of the starting materials as indicated by TLC). At the end of the reaction as justified by TLC analysis, the reaction mixture was evaporated at the reduced pressure to give the crude product which was purified on silica gel column by eluting the column with methanol and chloroform (1:49) solvent mixture to obtain the desired xyloside derivative. To the solution of a given fully protected xyloside (0.1 mmol), in dry methanol (3 mL) was added freshly prepared 0.5 M solution of CH3ONa (0.1 mL) in dry methanol, and the reaction mixture was stirred at room temperature for about 3 hours. Neutralization with H+ resin followed by concentration at reduced pressure gave a syrupy liquid, which was then purified on silica gel column to obtain the deprotected xyloside for biological evaluation.

To a solution containing protected xylosides (0.1 mmol), in dry methanol was added freshly prepared 0.5 M solution of CH3ONa (0.1 mL) in dry methanol, 3 mL methanol was stirred at room temperature for 3 hours. Neutralization with H+ resin followed by concentration at reduced pressure gave a syrupy liquid, which was purified on silica gel chromatography to give the desired deprotected triazole.

### Example 2

### General Procedure for Screening “Click” Xylosides

To determine whether the xylosides are able to prime the synthesis of glycosaminoglycans in cells in vitro experiments were performed using CHO pgsA-745. The cells were treated with appropriate primers in the presence of 35S—Na2SO4, glycosaminoglycans were purified and analyzed as described below. 1×10⁴ cells were plated per well in Ham's/F12 complete growth medium in a 24-well plate. The cells were incubated at 37° C. in a humidified incubator for 24 hours to a confluency of about 50%. The cells were washed with sterile PBS and replaced with 450 μL ham's/F12 containing 10% dialyzed FBS. Solutions containing a specific primer at 100× the final concentration were prepared. 5 μL of appropriate 100× primer were added to various wells to yield a final concentration of 100 μM. 50 μCi of 35S—Na2SO4 was also added to each well as tracer. The 24-well plates were placed back in the 37° C. incubator for 24 hours before the addition of 100 μL 6× pronase solution followed by incubation at 37° C. overnight.

### Example 3

### In Vitro Priming in Cell Culture

To determine whether the xylosides are able to prime the synthesis of glycosaminoglycans in cells, in vitro experiments were performed using CHO pgsA-745. The cells were treated with appropriate primers in the presence of 35S—Na2SO4, glycosaminoglycans were purified and analyzed as described below. 1×10⁴ cells were plated per well in Ham's/F12 complete growth medium in a 24-well plate. The cells were incubated at 37° C. in a humidified incubator for 24 hours to a confluency of about 50%. The cells were washed with sterile PBS and replaced with 450 uL ham's/F12 containing 10% dialyzed FBS. Serial dilutions of the primers at 100× the final concentration were prepared. 5 uL of appropriate 100× primer were added to various wells to yield a final concentration of 1, 10, 100 and 1000 uM respectively. 50 uCi of 35S—Na2SO4 was also added to each well as tracer. The 24-well plates were placed back in the 37° C. incubator for 24 hours before the addition of 100 uL 6× pronase solution followed by incubation at 37° C. overnight.

### Example 4

### Purification and Quantification of GAGs

The entire contents of the wells were transferred to a microcentrifuge tube and subjected to centrifugation at 16,000×g for 5 minutes. The supernatant was transferred to a fresh tube and half-a-volume of 0.016% Triton X-100 was added. The diluted supernant was loaded on 0.2 mL DEAE-sepharose column pre-equilibrated with 2 mL of 20 mM NaOAc buffer pH 6.0 containing 0.1 M NaCl and 0.01% Triton X-100 and the column was washed with 4 mL of buffer described above. The bound HS/CS was eluted using 1.2 mL elution buffer, 20 mM NaOAc, pH 6.0, containing 1 M NaCl. The extent of priming by the various xyloside primers was evaluated by quantifying the 35S-radioactivity incorporated in to the purified HS/CS elute by liquid scintillation. 50 uL of the various elutes were added to 5 mL of scintillation cocktail and the vials were counted in a scintillation counter in triplicate. The amount of radioactivity corresponds to the total glycosaminoglycan synthesis due to the primer.

### Example 5

### Determination of GAG Composition

To determine the composition of the HS and CS in the glycosaminoglycans synthesized, a small aliquot of the elute (300 uL) was diluted 10-fold and digested using Chondroitinase AC. The sample was subjected to precipitation to remove the disaccharide in the presence of cold chondroitin sulfate A and scintillation counted. This gives the amount of HS primed on the xylosides and the difference in the radioactivity corresponds to the amount of chondroitin sulfate in the purified glycoaminoglycan elute.

### Example 6

### Analysis of GAG Sulfation Density

To investigate the chain length/sulfation of the glycosaminoglycans primed by the various xylose primers the purified glycosaminoglycans were analyzed by HPLC with an inline radiodetector. 50 uL of elute was diluted five times with the HPLC solvent A (10 mM KH2P04, pH 6.0, 0.2% CHAPS) and loaded on to a weak anion exchange column, DEAE-3SW (TosoHaas Inc.,) and analyzed with the following elution profile. The sample was eluted from the column with a linear gradient of 0.2 M NaCl at 0 min to I M NaCl at 80 minutes at a flow rate of 1 mL per min. The radioactive glycosaminoglycans were detected by Radiomatic flo-one A505A radio-chromatography detector. The HPLC effluent was mixed with Ultima-Flo AP scintillation cocktail in a 2:1 ratio and detected in the flow scintillation detector.

### Example 7

### Chemical Characterization

The xylosides of the present invention were characterized by IR, NMR, and/or HRMS. The results were as follows.

The molecule shown in Formula A was characterized as follows: IR (neat) 3313 cm-1; 1H NMR (CD3OD): δ 8.12 (s, 1H), 6.93 (d, J=8.2 Hz, 1H), 6.70 (d, J=8.2 Hz, 1H), 6.68 (s, 1H), 5.51 (d, J=9.0 Hz, 1H), 4.40 (d, J=5.5 Hz, 2H), 3.78 (s, 3H), 2.20 (t, J=7.8 Hz, 2H), 1.64 (br m, 2H), 2.67-2.72 (m, 4H), 1.74-1.78 (m, 4H); HRMS (ESI): calculated for C₁₈H₂₃N₃O₅+Na 384.1535, found 384.1587.

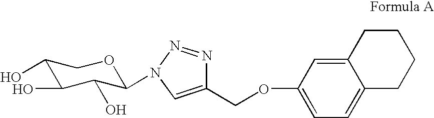

The molecule shown in Formula B was characterized as follows: IR (neat) 3340 cm-¹; 1H NMR (CD₃OD): δ8.24 (s, 1H), 7.26-7.30 (m, 2H), 6.93-7.02 (m, 3H), 5.52 (d, J=9.6 Hz, 1H), 5.17 (s, 2H), 4.00 (dd, J=5.1, 11.3 Hz, 1H), 3.90 (t, J=8.8 Hz, 1H), 3.63-3.71 (m, 1H), 3.44-3.53 (m, 2H); ¹³C NMR (CD₃OD): δ 158.6, 144.0, 129.4, 123.3, 121.1, 114.6, 89.1, 77.4, 72.7, 69.5, 68.7, 63.2, 61.1; CIMS (NH3), m/z 307.9; HRMS (ESI): calculated for C₁₄H₁₇N₃0₅+Na 330.1066, found 330.0534.

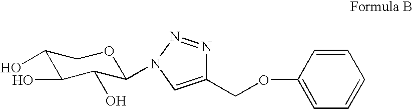

The molecule shown in Formula C was characterized as follows: ¹H NMR (CD₃OD): δ 8.27 (s, 1H), 7.25-7.29 (m, 2H), 6.92-7.01 (m, 3H), 6.13 (d, J=2.8 Hz, 1H), 5.17 (s, 2H), 4.15 (t, J=5.4 Hz, 1H), 4.08 (dd, J=3.2, 12.0 Hz, 1H), 3.85-3.92 (m, 2H), 3.64-3.67 (m, 1H); ¹³C NMR (CD₃OD): δ 155.7, 140.3, 126.4, 121.7, 118.1, 111.7, 82.7, 68.0, 67.4, 66.0, 64.9, 58.1; HRMS (ESI): calculated for C₁₄H₁₇N₃0₅+Na 330.1066, found 330.0534.

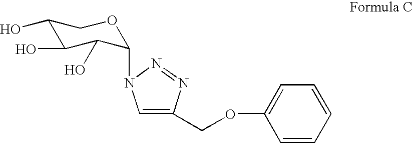

The molecule shown in Formula D was characterized as follows: IR (neat) 3336 cm-¹; ¹H NMR (CD₃OD): δ 8.23 (s, 1H), 6.96-7.01 (m, 4H), 5.21 (d, J=9.6 Hz, 1H), 5.15 (s, 2H), 4.01 (dd, J=5.6, 11.6 Hz, 1H), 3.89 (t, J=8.8 Hz, 1H), 3.64-3.70 (m, 1H), 3.44-3.51 (m, 2H); HRMS (ESI): calculated for C₁₄H₁₆FN₃O₅+Na 348.0972, found 348.2255

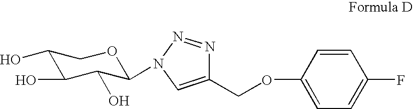

The molecule shown in Formula E was characterized as follows: IR (neat) 3401 cm-¹; ¹H NMR (CD₃OD): δ 8.27 (s, 1H), 7.53-7.57 (m, 4H), 7.39 (t, J=7.6 Hz, 2H), 7.27 (t, J=7.6 Hz, 1H), 7.08-7.11 (m, 2H), 5.53 (d, J=8.8 Hz, 1H), 5.23 (s, 2H), 4.01 (dd, J=5.6, 11.4 Hz, 1H), 3.96 (t, J=8.8 Hz, 1H), 3.62-3.71 (m, 1H), 3.44-3.52 (m, 2H); HRMS (ESI): calculated C₂₀H₂₁N₃O₅+Na 406.1379, found 406.1258

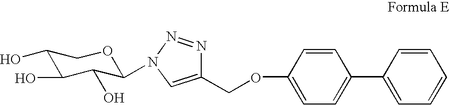

The molecule shown in Formula F was characterized as follows: IR (neat) 3370 cm-¹; ¹H NMR (CD₃OD): δ 8.27 (s, 1H), 7.48-7.57 (m, 6H), 7.39 (d, J=8.4 Hz, 2H), 7.08-7.11 (m, 2H), 5.53 (d, J=9.2 Hz, 1H), 5.23 (s, 2H), 3.91 (dd, J=5.6, 11.0 Hz, 1H), 3.68-3.72 (m, 1H), 3.57 (t, J=9.6 Hz, 1H), 3.43-3.48 (m, 2H); HRMS (ESI): calculated for C₂₂H₂₁N₃0₅+Na 462.0665, found 461.8000.

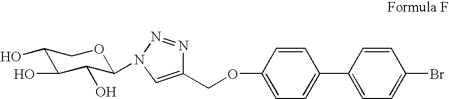

The molecule shown in Formula G was characterized as follows: IR (neat) 3293 cm-¹; ¹H NMR (CD₃OD): δ 8.55-8.70 (m, 2H), 8.39 (s, 1H), 8.28 (d, J=7.4 Hz, 1H), 7.82 (d, J=7.4 Hz, 1H), 7.48-7.67 (m, 4H), 7.28 (s, 1H), 5.58 (d, J=9.2 Hz, 1H), 5.45 (s, 2H), 4.01 (dd, J=5.6, 11.2 Hz, 1H), 3.96 (t, J=9.2 Hz, 1H), 3.68-3.72 (m, 1H), 3.46-3.55 (m, 2H); HRMS (ESI): calculated for C₂₂H₂₁N₃O₅+Na 430.1379, found 430.1333.

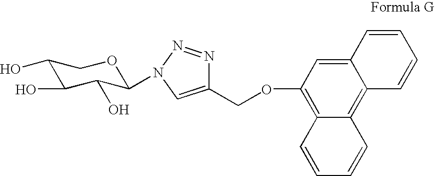

The molecule shown in Formula H was characterized as follows: IR (neat) 3409 cm-1; 1H NMR (CD₃OD): δ 8.31-8.33 (m, 1H), 8.30 (s, 1H), 8.16 (brs, 1H), 7.55-7.57 (m, 1H), 7.37-7.40 (m, 1H), 5.53 (d, J=9.2 Hz, 1H), 5.28 (s, 2H), 4.01 (dd, J=5.6, 11.4 Hz, 1H), 3.89 (t, J=9.2 Hz, 1H), 3.64-3.70 (m, 1H), 3.44-3.52 (m, 2H); HRMS (ESI): calculated for C₁₃H₁₆N₄O₅+H 309.1199, found 309.0867.

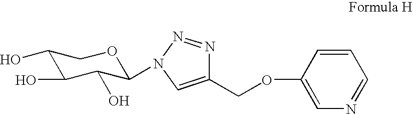

The molecule shown in Formula I was characterized as follows: 1H NMR (CD_{3O}D): δ 8.35-8.30 (m, 1H), 8.33 (s, 1H), 8.16 (brs, 1H), 7.55-7.57 (m, 1H), 7.37-7.40 (m, 1H), 6.14 (d, J=3.2 Hz, 1H), 5.28 (s, 2H), 4.15 (t, J=5.2 Hz, 1H), 4.08 (dd, J=3.2, 11.8 Hz, 1H), 3.85-3.92 (m, 2H), 3.64-3.67 (m, 1H); HRMS (ESI): calculated for C₁₃H₁₆N₄O₅+H 309.1199, found 309.0867.

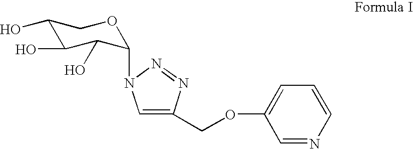

The molecule shown in Formula J was characterized as follows: IR (neat) 3409 cm-1; 1H NMR (CD3OD): δ 8.31 (s, 1H), 8.22 (d, J=7.0 Hz, 2H), 7.18 (d, J=7.0 Hz, 2H), 5.54 (d, J=8.8 Hz, 1H), 5.32 (s, 2H), 4.00 (dd, J=5.6, 11.2 Hz, 1H), 3.90 (t, J=8.8 Hz, 1H), 3.65-3.71 (m, 1H), 3.44-3.52 (m, 2H); HRMS (ESI): calculated for C₁₄H₁₆N₄O₇ 352.1019, found 352.8739.

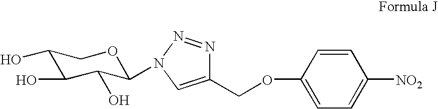

The molecule shown in Formula K was characterized as follows: IR (neat) 3424 cm-1; 1H NMR (CD₃OD): δ 8.15 (s, 1H), 7.76 (s, 1H), 5.55 (d, J=9.2 Hz, 1H), 4.00 (dd, J=5.2, 11.4 Hz, 1H), 3.90 (t, J=8.8 Hz, 1H), 3.65-3.70 (m, 1H), 3.44-3.52 (m, 2H).

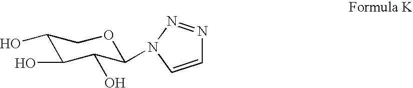

The molecule shown in Formula L was characterized as follows: 1H NMR (CD₃OD): δ 8.21 (s, 1H), 7.74 (s, 1H), 6.15 (d, J=2.8 Hz, 1H), 4.16 (t, J=5.9 Hz, 1H), 4.07 (dd, J=3.2, 11.8 Hz, 1H), 3.85-3.91 (m, 2H), 3.65-3.67 (m, 1H).

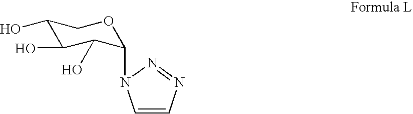

The molecule shown in Formula M was characterized as follows: 1H NMR (CD₃OD): δ 8.10 (s, 1H), 5.55 (d, J=9.2 Hz, 1H), 4.70 (s, 2H), 4.00 (dd, J=5.6, 11.2 Hz, 1H), 3.88 (t, J=9.2 Hz, 1H), 3.64-3.70 (m, 1H), 3.43-3.57 (m, 2H); 13C NMR (CD₃OD): δ 122.3, 89.1, 77.4, 72.7, 69.5, 68.7, 63.2, 55.2.

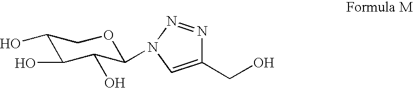

The molecule shown in Formula N was characterized as follows: IR (neat) 3390 cm-1; 1H NMR (CD₃OD): δ 8.52 (s, 1H), 7.84 (d, J=8.4 Hz, 2H), 7.32-7.45 (m, 3H), 5.55 (d, J=9.3 Hz, 1H), 4.04 (dd, J=5.6, 11.2 Hz, 1H), 3.94 (t, J=9.3 Hz, 1H), 3.67-3.73 (m, 1H), 3.47-3.54 (m, 2H); 13C NMR (CD₃OD): δ 147.7, 130.4, 128.8, 128.3, 125.5, 120.1, 89.2, 77.4, 72.8, 69.5, 68.7; HRMS (ESI): calculated for C₁₃H₁₅N₃O₄+Na 300.0960, found 300.0953.

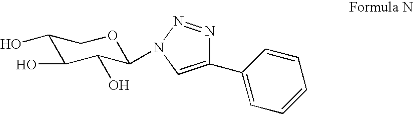

The molecule shown in Formula O was characterized as follows: IR (neat) 3417 cm-1; 1H NMR (CD₃OD): δ 8.29 (s, 1H), 7.74-7.94 (m, 3H), 7.30-7.45 (m, 3H), 7.14-7.18 (m, 1H), 5.54 (d, J=9.2 Hz, 1H), 5.30 (s, 2H), 4.01 (dd, J=5.6, 11.2 Hz, 1H), 3.91 (t, J=9.2 Hz, 1H), 3.65-3.71 (m, 1H), 3.44-3.52 (m, 2H); HRMS (ESI): calculated for C₁₈H₁₉N₃O₅+Na 380.1222, found 380.1171.

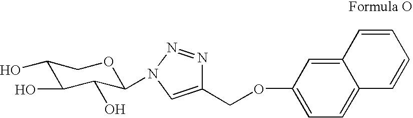

The molecule shown in Formula P was characterized as follows: IR (neat) 3305 cm-1; 1H NMR (CD₃OD): δ 8.30 (s, 1H), 7.62-7.67 (m, 1H), 7.70-7.74 (m, 2H), 7.50-7.53 (m, 1H), 7.41 (m, 1H), 7.20-7.23 (m, 1H), 5.53 (d, J=9.2 Hz, 1H), 5.30 (s, 2H), 4.01 (dd, J=5.6, 11.4 Hz, 1H), 391 (t, J=8.8 Hz, 1H), 3.65-3.71 (m, 1H), 3.44-3.52 (m, 2H); HRMS (ESI): C₁₈H₁₈BrN₃O₅+H 436.0508, found 435.9250.

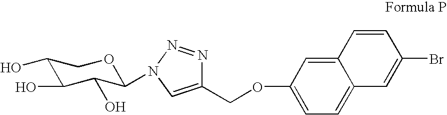

The molecule shown in Formula Q was characterized as follows: IR (neat) 3382 cm-1; 1H NMR (400 MHz, CD₃OD) δ 8.17 (s, 1H), 7.8-7.84 (m, 4H), 7.43-7.48 (m, 3H), 5.52 (d, J=9.0 Hz, 1H), 4.73 (s, 2H), 4.69 (s, 2H), 4.01 (t, J=9.0 Hz, 1H), 3.90 (dd, J=5.6, 9.4 Hz, 1H), 3.65-3.71 (m, 1H), 3.44-3.52 (m, 2H); HRMS (ESI): calculated for C₁₉H₂₁N₃O₅+Na 394.1370, found 394.0724.

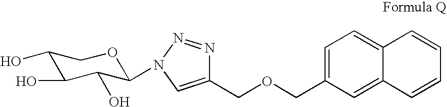

The molecule shown in Formula R was characterized as follows: IR (neat) 3347 cm-1; 1H NMR (400 MHz, CD₃OD): δ 8.15 (s, 1H), 7.25-7.36 (m, 4H), 5.51 (d, J=9.4 Hz, 1H), 4.76 (s, 2H), 4.59 (s, 2H), 4.01 (t, J=5.6 Hz, 1H), 3.89 (dd, J=xx, 9.4 Hz, 1H), 3.66-3.70 (m, 1H), 3.44-3.51 (m, 2H); HRMS (BSI): calculated for C₁₆H₂₁N₃O₅+Na 374.1328, found 374.3030.

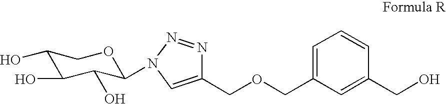

The molecule shown in Formula S was characterized as follows: IR (neat) 3316 cm-1; 1H NMR (CDCl₃): δ 8.25 (s, 1H), 6.94-7.41 (m, 1H), 5.52 (d, J=9.2 Hz, 1H), 5.167 (s, 2H), 4.01 (dd, J=5.6, J=11.4 Hz, 1H), 3.89 (t, J=9.4 Hz, 1H), 3.34-3.58 (m, 1H), 3.34-3.49 (m, 2H); HRMS (BSI): calculated for C₁₄H₁₆BrN₃O₅+Na 408.0171, found 408.1097.

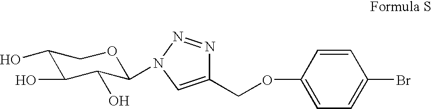

The molecule shown in Formula T was characterized as follows: IR (neat) 3351 cm-1; 1H NMR (CDCl₃): δ 8.25 (s, 1H), 6.83-7.59 (m, 4H), 5.32 (d, J=9.3 Hz, 1H), 5.16 (s, 2H), 4.01 (dd, J=5.6, 10.3 Hz, 1H), 3.89 (t, 1H), 3.46-3.51 (m, 2H); HRMS (ESI): calculated for C₁₄H₁₆IN₃O₅+NA 546.0032, found 455.9333.

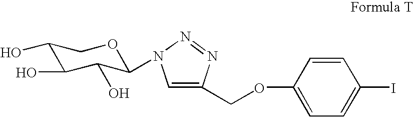

The molecule shown in Formula U was characterized as follows: 1H NMR (CDCl₃): δ 8.24 (s, 1H), 6.82-6.94 (m, 4H), 6.12 (d, J=2.7 Hz, 1H); 5.10 (s, 2H), 4.16 (t, J=5.6 Hz, 1H), 4.07 (dd, J=2.7, 11.6 Hz, 1H), 3.86-3.91 (m, 2H), 3.73 (s, 3H), 3.65-3.67 (m, 1H); HRMS (ESI): calculated for C₁₅H₁₉N₃O₅+Na 338.1352, found 337.9333

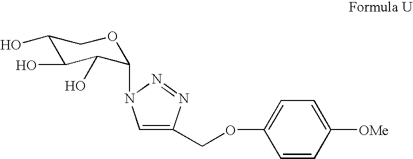

The molecule shown in Formula V was characterized as follows: 1H NMR (CDCl₃): δ 8.20 (s, 1H), 6.82-6.95 (m, 4H), 5.52 (d, J=9.0 Hz, 1H), 5.11 (s, 2H), 4.01 (dd, J=5.6, 11.0 Hz, 1H), 3.89 (t, J=9.0 Hz, 1H), 3.73 (S 3H), 3.64-3.70 (m, 1H), 3.44-3.52 (m, 2H); HRMS (ESI): calculated for C₁₅H₁₉N₃O₅+Na 338.1352, found 337.9333.

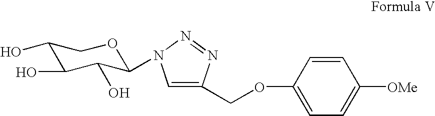

The molecule shown in Formula W was characterized as follows: IR (neat) 3336 cm-1; 1H NMR (CDCl₃): δ 8.25 (s, 1H), 6.98-7.28 (m, 4H), 5.52 (d, J=9.0 Hz, 1H), 5.17 (s, 2H), 4.01 (dd, J=5.6, 11.3 Hz, 1H), 3.89 (t, J=5.6 Hz, 1H), 3.64-3.70 (m, 1H), 3.48-3.64 (m, 2H); HRMS (ESI): calculated for C₁₄H₁₆ClN₃O₅+Na 364.0676, found 364.0999.

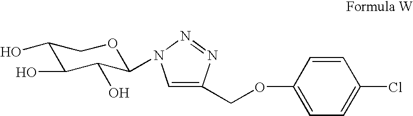

The molecule shown in Formula X was characterized as follows: IR (neat) 3343 cm-1; 1H NMR (CDCl₃): δ 8.15 (s, 1H), 7.24, 7.27 (m, 1H), 5.51 (d, J=9.0 Ha, 1H), 4.68 (s, 2H), 4.58 (s, 2H), 4.01 (dd, J=5.6, 11.4 Hz, 1H), 3.89 (t, J=9.0 Hz, 1H), 3.64-3.70 (m, 1H), 3.44-3.51 (m, 2H); HRMS (ESI): calculated for C₁₅H₁₉N₃O₅+H 322.1403, found 321.9333.

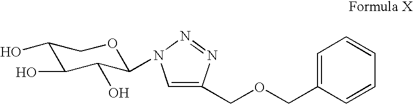

The molecule shown in Formula Y was characterized as follows: 1H NMR (CDCb): δ 8.19 (s, 1H), 7.26-7.36 (m, 4H), 6.12 (d, J=3.2 Hz, 1H), 4.65 (s, 2H), 4.58 (s, 2H), 4.15 (t, J=5.0 Hz, 1H); 4.07 (dd, J=2.7, 12.1 Hz, 1H), 3.85-3.91 (m, 2H), 3.63-3.67 (m, 2H); HRMS (ESI): calculated for C₁₅H₁₉N₃O₅+H 322.1403, found 321.9333.

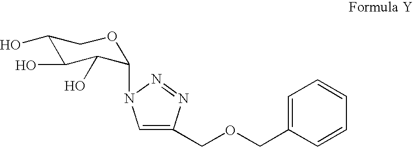

The molecule shown in Formula Z was characterized as follows: IR (neat) 3328 cm-1; 1H NMR (CD₃OD): δ 7.84 (s, 1H), 7.17-7.30 (m, 5H), 5.45 (d, J=8.9 Hz, 1H), 4.05 (s, 2H), 3.98 (dd, J=5.6, 11.4 Hz, 1H), 3.86 (t, J=9.2 Hz, 1H), 3.62-3.68 (m, 1H), 3.41-3.50 (m, 2H); HRMS (ESI): calculated for C₁₄H₁₇N₃O₄+H 292.1297, found 292.0000.

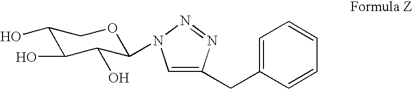

The molecule shown in Formula AA was characterized as follows: IR (neat) 3343 cm-1, 1H NMR (CD3OD): δ 8.10 (s, 1H), 5.50 (d, J=9.2 Hz, 1H), 4.64 (s, 2H), 4.01 (dd, J=5.6, 11.4 Hz, 1H), 3.88 (t, J=9.6 Hz, 1H), 3.64-3.70 (m, 1H), 3.43-3.51 (m, 2H), 1.92-1.97 (m, 2H), 1.71-1.78 (m, 2H), 1.52-1.56 (m, 1H), 1.26-1.33 (m, 5H); HRMS (ESI): calculated for C₁₄H₂₃N₃O₅+Na 336.1535, found 336.1333.

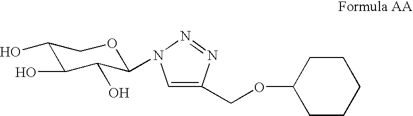

The molecule shown in Formula AB was characterized as follows: IR (neat) 3340 cm-1; 1H NMR (CD₃OD): δ 8.16 (s, 1H), 7.35-7.40 (m, 2H), 7.04-7.08 (m, 2H), 5.51 (d, J=9.2 Hz, 1H), 4.65 (s, 2H), 4.55 (s, 2H), 4.01 (dd, J=5.6, 11.4 Hz, 1H), 3.89 (t, J=9.2 Hz, 1H), 3.64-3.71 (m, 1H), 3.44-3.52 (m, 2H); HRMS (ESI): calculated for C₁₅H₁₈FN₃O₅+Na 362.1128, found 362.1333.

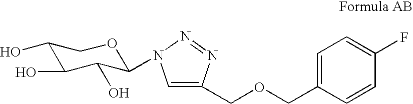

The molecule shown in Formula AC was characterized as follows: 1H NMR (CD₃OD): δ 3.44-3.52 (m, 4H), 3.64-3.71 (m, 2H), 3.90 (dd, 2H, J=9.6 Hz, 5.4 Hz), 4.00 (t, 2H, J=5.6 Hz), 4.586 (s, 4H), 4.65 (s, 4H), 5.52 (d, 2H, J=9.4 Hz), 7.287.37 (m, 4H), 8.17 (s, 2H).

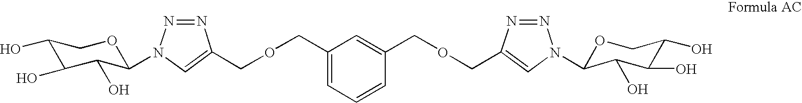

The molecule shown in Formula AD was characterized as follows: 1H NMR (CD₃OD): δ 8.07 (s, 1H), 8.06 (d, J=8.2 Hz, 1H), 5.92 (d, J=4.3 Hz, 1H), 5.79 (d, J=8.2 Hz, 1H), 5.46 (d, J=9.0 Hz, 1H), 5.20 (s, 2H), 4.13-4.88 (m, 2H), 3.96-4.02 (m, 2H), 3.83-3.87 (m, 2H), 3.71-3.75 (m, 1H), 3.59-3.69 (m, 1H), 3.44-3.49 (m, 2H).

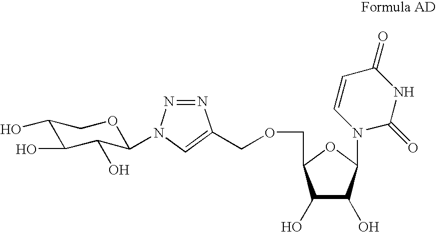

The molecule shown in Formula AE was characterized as follows: 1H NMR (CD₃OD): δ 8.06 (s, 1H), 8.00 (d, J=8.2 Hz, 1H), 6.28 (t, J=6.6 Hz, 1H), 5.79 (d, J=8.2 Hz, 1H), 5.46 (d, J=9.0 Hz, 1H), 5.20 (s, 2H), 4.36-4.39 (m, 1H), 3.98 (dd, J=5.4, 11.3 Hz, 1H), 3.90-3.92 (m, 1H), 3.85 (t, J=9.2 Hz, 1H), 3.62-3.79 (m, 3H), 3.71-3.9 (m, 2H), 2.82-2.31 (m, 1H), 2.19-2.24 (m, 1H).

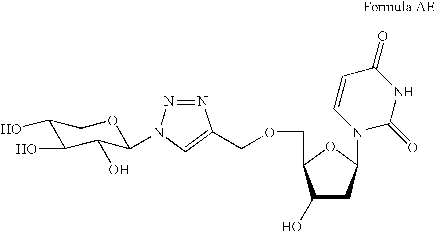

### Example 9

### Biological Activity

a) To determine whether the xylosides are able to prime the synthesis of glycosaminoglycans in cells in vitro experiments were performed using CHO pgsA-745. The cells were treated with appropriate primers in the presence of ³⁵S—Na₂SO₄, glycosaminoglycans were purified and analyzed as described below. 1×10⁴ cells were plated per well in Ham's/F12 complete growth medium in a 24-well plate. The cells were incubated at 37 C in a humidified incubator for 24 hours to a confluency of about 50%. The cells were washed with sterile PBS and replaced with 450 uL ham's/F12 containing 10% dialyzed FBS. Serial dilutions of the primers at 100× the final concentration were prepared. 5 uL of appropriate 100× primer were added to various wells to yield a final concentration of 1, 10, 100 and 1000 uM respectively. 50 uCi of ³⁵S—Na₂SO₄ was also added to each well as tracer. The 24-well plates were placed back in the 37 C incubator for 24 hours before the addition of 100 uL 6× pronase solution followed by incubation at 37 C overnight.

The entire contents of the wells were transferred to a microcentrifuge tube and subjected to centrifugation at 16,000×g for 5 minutes. The supernatant was transferred to a fresh tube and half-a-volume of 0.016% Triton X-100 was added. The diluted supernatant was loaded on 0.2 mL DEAE-sepharose column pre-equilibrated with 2 mL of 20 mM NaOAc buffer pH 6.0 containing 0.1 M NaCl and 0.01% Triton X-100 and the column was washed with 4 mL of buffer described above. The bound HS/CS was eluted using 1.2 mL elution buffer, 20 mM NaOAc, pH 6.0 containing 1 M NaCl. The extent of priming by the various xyloside primers was evaluated by quantifying the ³⁵S -radioactivity incorporated in to the purified HS/CS elute by liquid scintillation. 50 uL of the various elutes were added to 5 mL of scintillation cocktail and the vials were counted in a scintillation counter in triplicate. The amount of radioactivity corresponds to the total glycosaminoglycan synthesis due to the primer.

b) To determine the composition of the HS and CS in the glycosaminoglycans synthesized, a small aliquot of the elute (300 uL) was diluted 10-fold and digested using Chondroitinase AC. The sample was subjected to precipitation to remove the disaccharide in the presence of cold chondroitin sulfate A and scintillation counted. This gives the amount of HS primed on the xylosides and the difference in the radioactivity corresponds to the amount of chondroitin sulfate in the purified glycoaminoglycan elute.

c) To investigate the chain length/sulfation of the glycosaminoglycans primed by the various xylose primers the purified glycosaminoglycans were analyzed by HPLC with an inline radiodetector. 50 uL of elute was diluted five times with the HPLC solvent A (10 mM KH₂PO₄, pH 6.0, 0.2% CHAPS) and loaded on to a weak anion exchange column, DEAE-3SW (TosoHaas Inc.,) and analyzed with the following elution profile. The sample was eluted from the column with a linear gradient of 0.2 M NaCl at 0 min to I M NaCl at 80 minutes at a flow rate of 1 mL per mM. The radioactive glycosaminoglycans were detected by Radiomatic flo-one A505A radio-chromatography detector. The HPLC effluent was mixed with Ultima-Flo AP scintillation cocktail in a 2:1 ratio and detected in the flow scintillation detector.

It has been found that the priming activity of primers with various aglycone moieties can vary. At 100 uM concentration the phenyl, tetrahydronaphthyl and naphthyl moieties primed very well. The methylfuran moiety had lower than phenyl aglycone. The presence of pyridine and phenanthrene rings in the aglycone reduced their priming ability. Also, it has to be noted that primers with only the triazole in the aglycone had reduced priming ability.

The effect of alpha- and beta-linked xylose was also investigated. The alpha- and beta-anomers of xyloside primers with triazole only, phenyl and pyridyl aglycone moieties were characterized for their priming activity. At all concentrations, the beta-anomer primed better than the alpha-anomer.

It was found that various substitutions on the phenyl ring leads to subtle changes in the priming activity. Comparing the priming activity of phenyl aglycone with primers that have various substitution in their phenyl shows very similar priming ability. The fluoro substitution on the phenyl increases the priming activity by a small amount. The chloro and bromo substitutions do not significantly change the priming activity from the fluoro substituted primer. The iodo, methoxy and nitro substitutions decrease the priming activity of the xylosides.

The substitution of bromine in the biphenyl aglycone does not significantly alter the priming of glycosaminoglycans. In the case of naphthyl aglycone, the bromo substitution reduced the priming activity of the primer at 100 uM concentration, However, at 10 uM the priming ability of the xylosides are not significantly different

The effect of the presence of multiple xylose in a single primer molecule was also determined. At 100 uM, the single xylose containing primer was a very effective primer. The presence of three xyloses in a primer increased the priming ability by 50% compared the single xylose primer. The trixyloside primer was used as the acetate protected molecule to increase its ability to penetrate the cell membrane.

Three xyloside primers were prepared with thio/sulfo linkage to aglycone moieties. All three compounds showed significant priming activities at 100 uM; the sulfone-phenyl had about 60% activity as the thio-phenyl and thio-pyridyl primers. However, the sulfone-phenyl xyloside better activity at concentration as low as 1 uM. Replacement of the oxygen with a sulfur atom decreases the priming ability of xyloside.

The comparative glycosaminoglycan priming activity of all the xyloside primers used in the assay at 0.1 uM had reduced priming activity. At 1 uM, the sulfone-phenyl xyloside had the highest activity than any of the primers. The priming ability was four times that of the next highest priming activity seen at this concentration and was comparable to the activity seen for other primers that primed very well at 100 uM.

At 10 uM, tetrahydronaphthyl and the S-glycosides, thiophenyl and sulfone-phenyl have significantly higher priming activity than other xylosides.

Most primers have good priming ability at 100 uM concentration. All of the phenyl, biphenyl, naphthyl, aglycones with the triazole/thio/sulfone/C-linkage act as good primers. However, the pyridine, bromonaphthyl and phenanthrene containing xylosides do not prime as effectively. The two xylosides that have the highest priming, activity are triazole primers with phenyl and benzyl alcohol as aglycones.

Half of the glycosaminoglycans primed by triazole-O-phenyl and its fluoro-substituted derivative are HS. However, substitution with chloro, bromo, iodo, methoxy, or nitro groups decreases the composition by 10 to 30%. The substitution of the above phenyl by tetrahydronaphthyl, naphthyl, biphenyl, or phenanthrene also leads to decrease in the priming of HS. On the other hand, when the oxygen atom in the triazole-O-naphthyl is changed to a sulfur atom, the xyloside predominantly primes HS. Also, ±60% HS is primed by the following xylosides; two xyloses linked together by triazole and triazole-phenyl. The highest composition of HS (−72%) is primed by C-(2-naphthol)-xyloside.

### Example 10

### HPLC Analysis

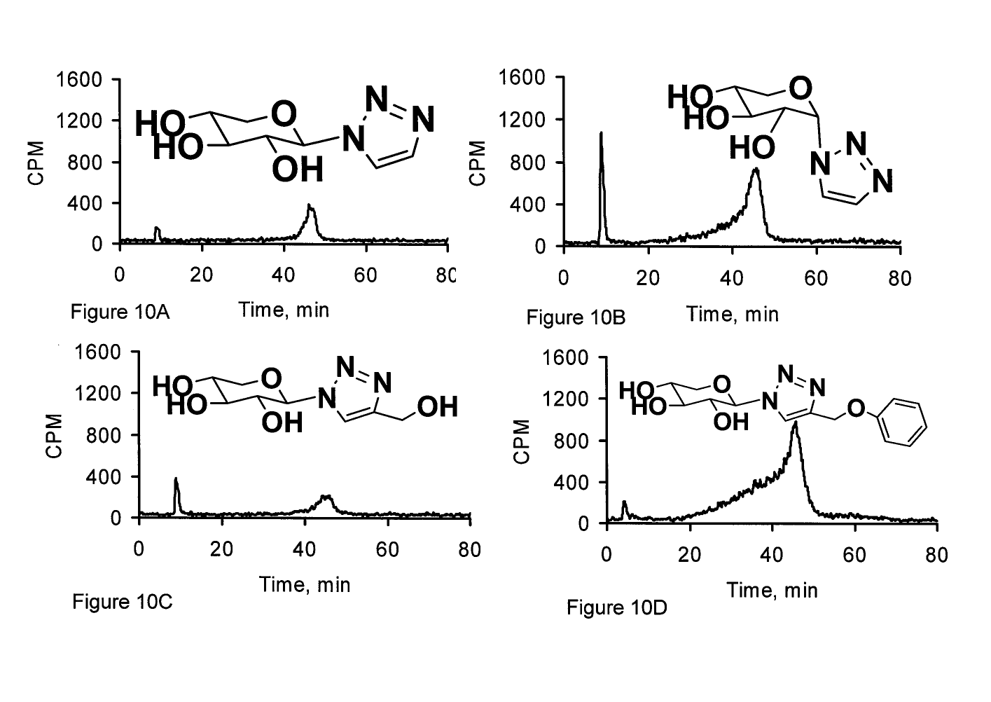

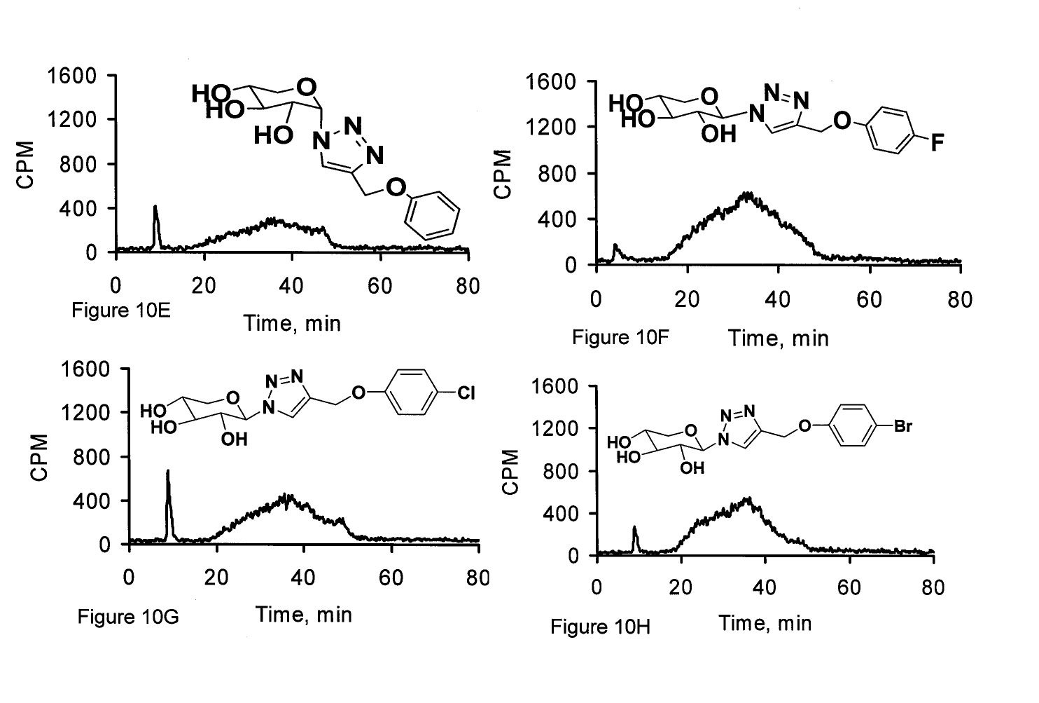

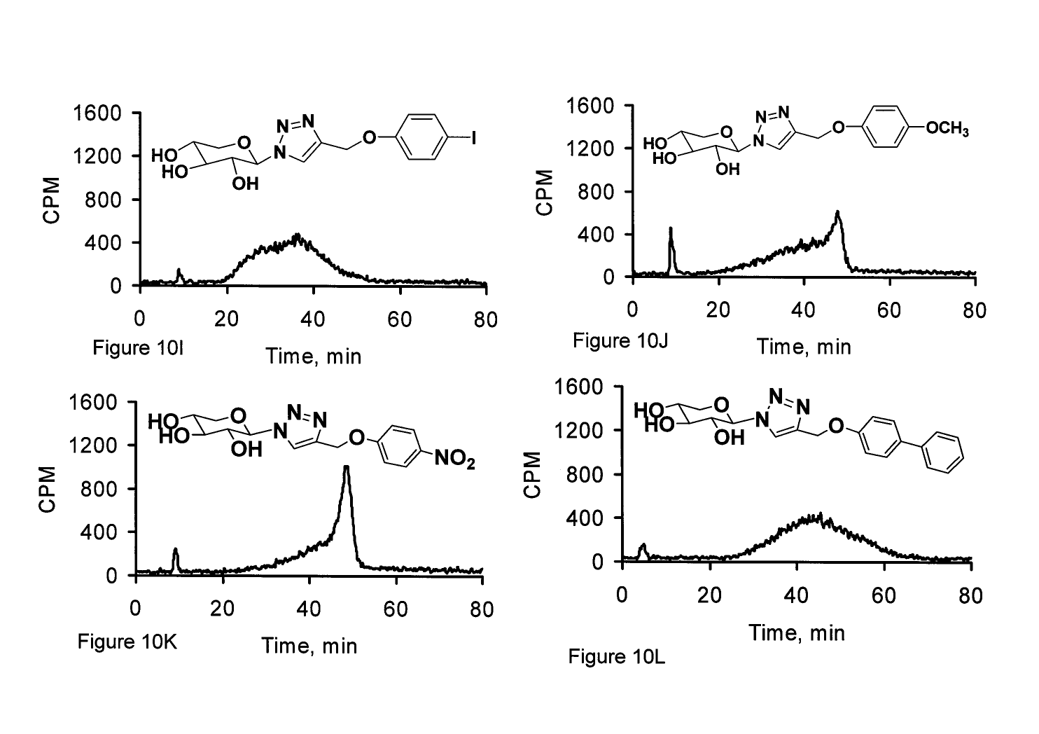

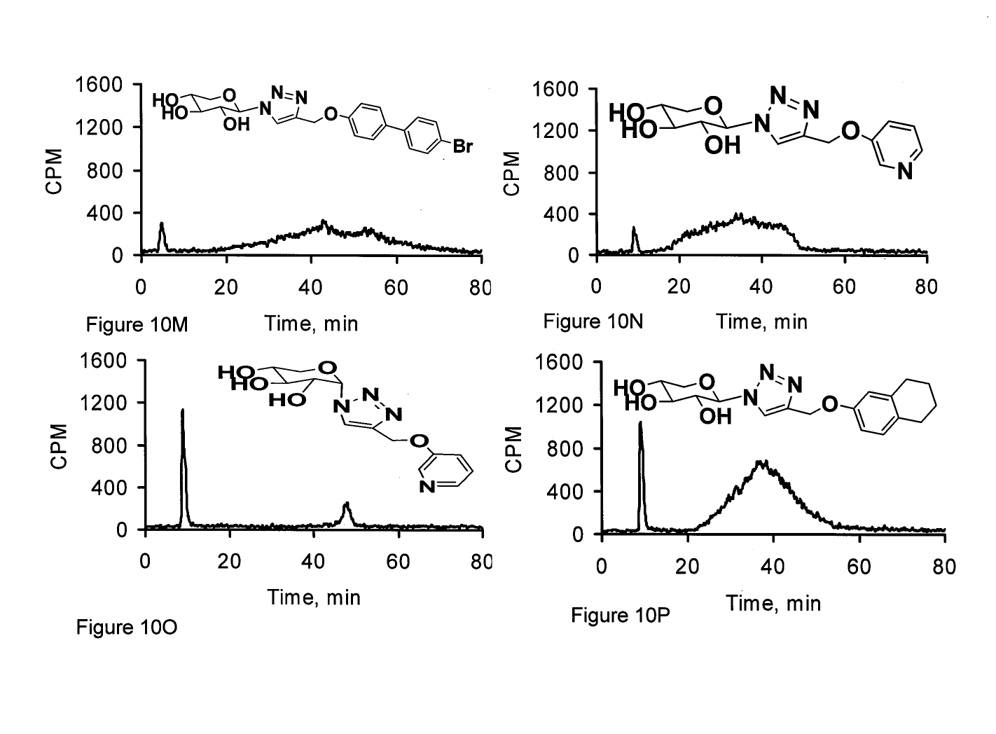

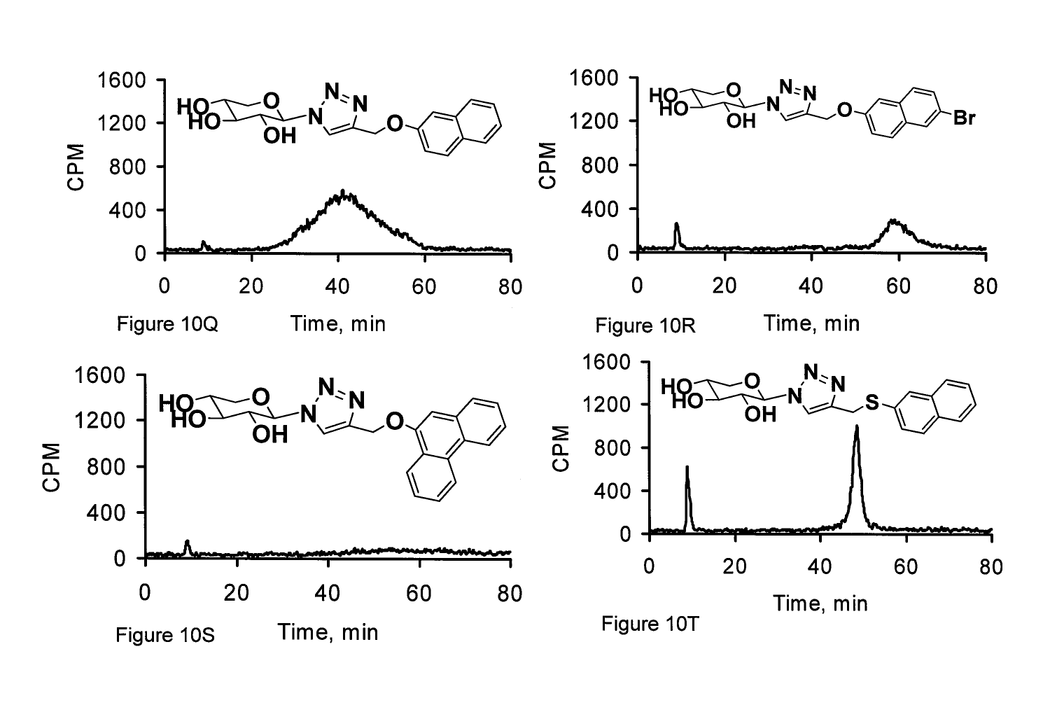

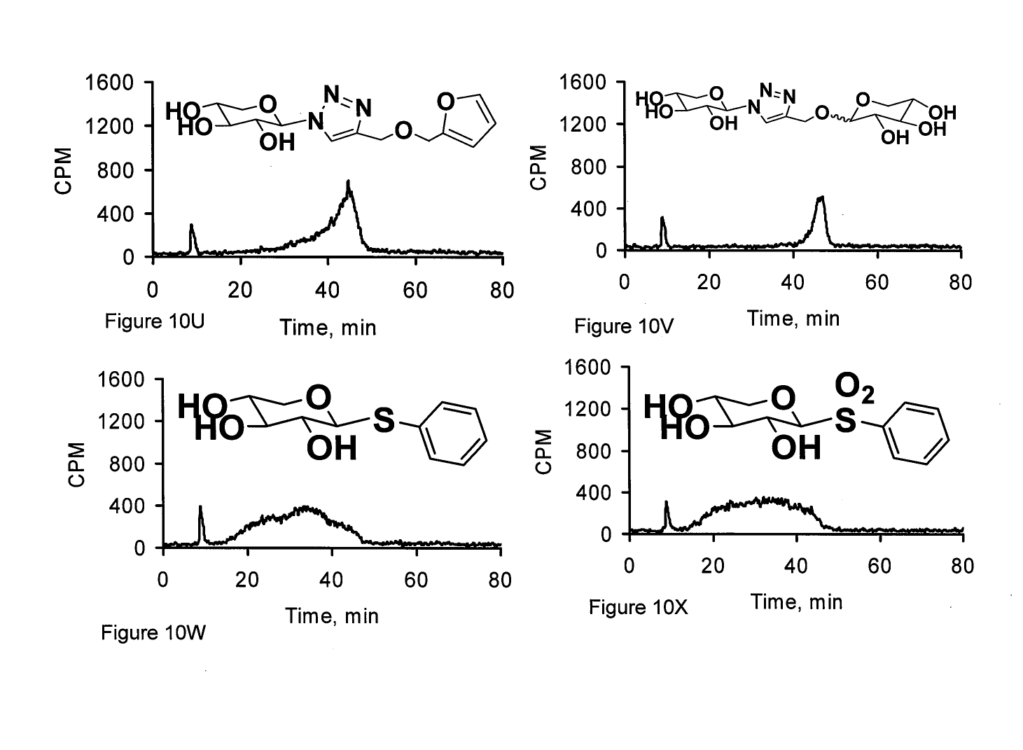

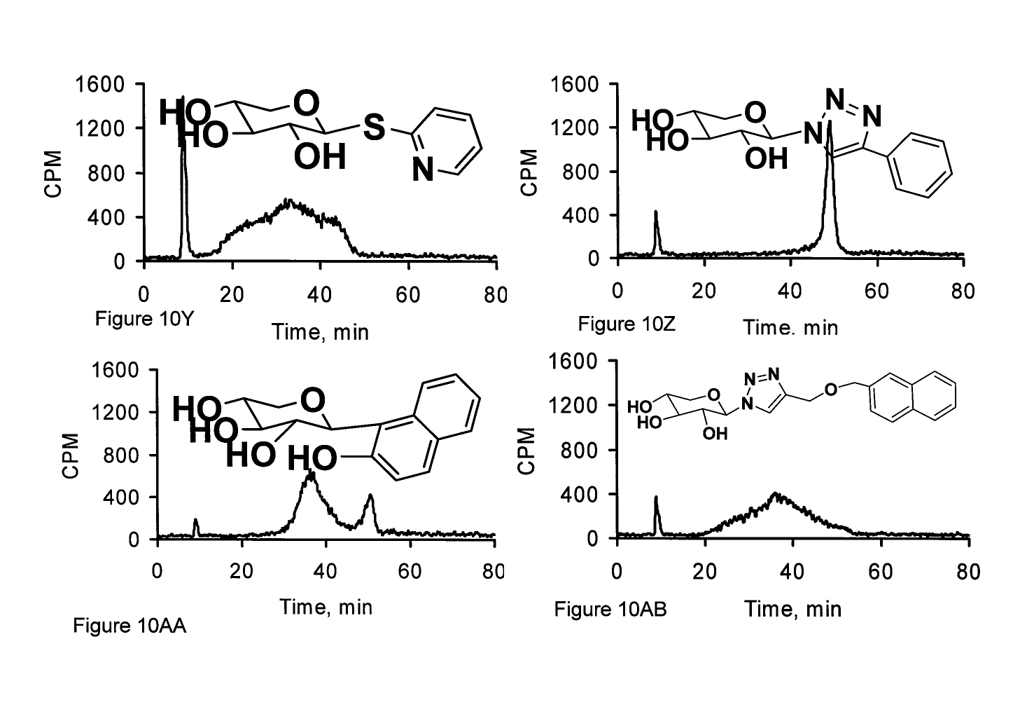

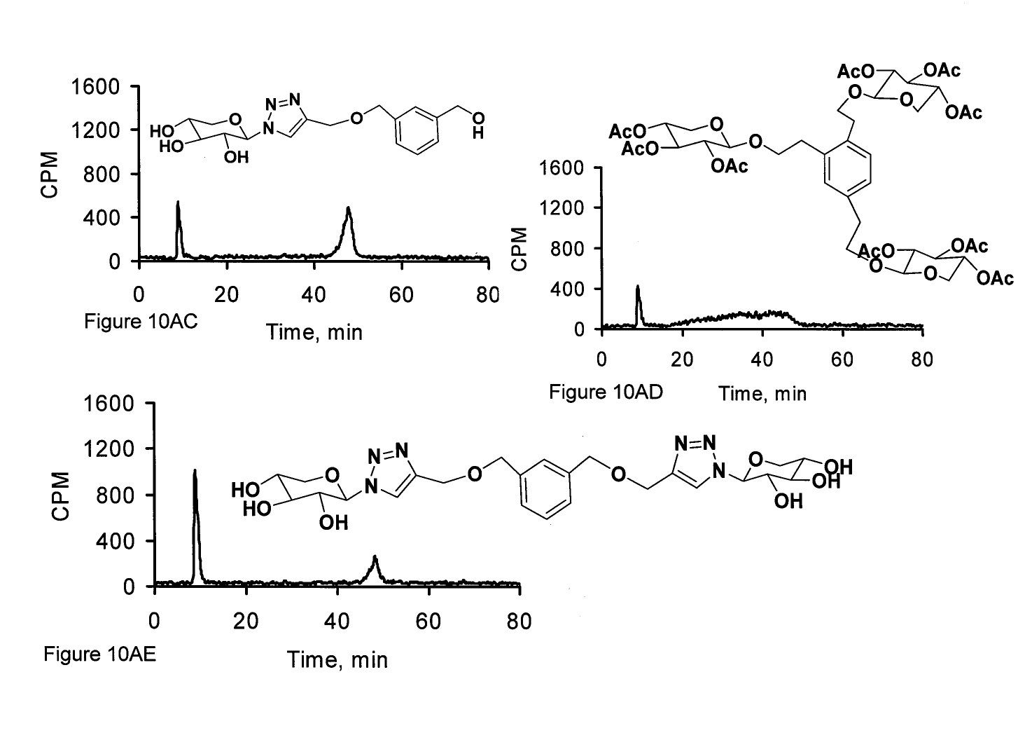

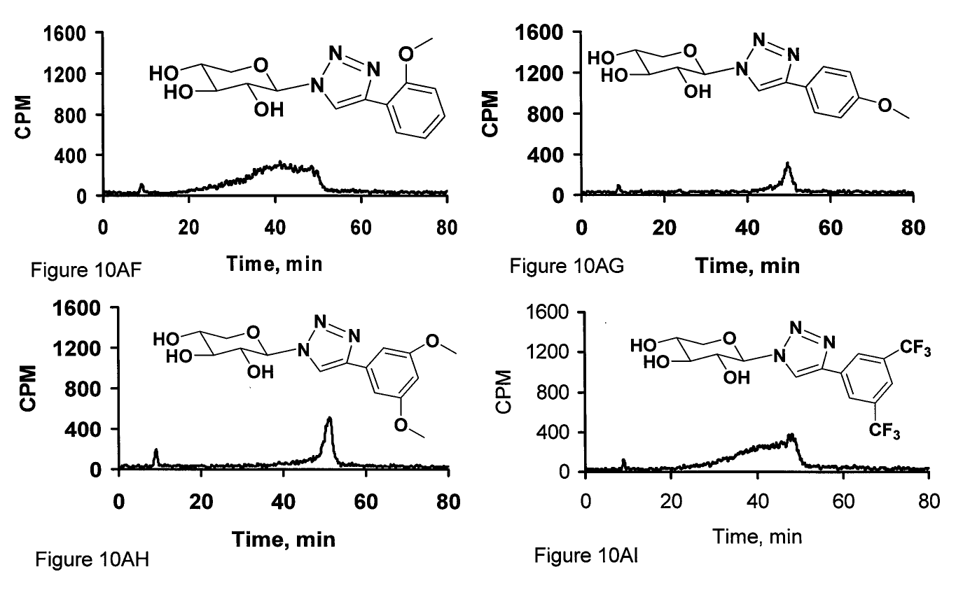

The HPLC analyses of the purified glycosaminoglycans from cells treated with 100 uM xylosides are shown above. For some of the 100 uM samples which did not have high enough radioactivity for the radiodetection, the 1 mM sample or 10 uM sample was used. In addition, for samples that had very good priming ability at 10 uM or 1 mM treatment analyses were performed in addition to the 100 uM samples. The HPLC profile varied for the samples depending on the aglycone moiety present on the xylosides. In addition to the O-linked phenyl and nitrophenyl xylosides, the S-linked thionaphthyl and C-linked phenyl xylosides showed a sharper peak indicating more uniform sulfation/chain length of the primed glycosaminoglycans. All the other substituted phenyl, biphenyl and pyridyl aglycones showed broader HPLC profile indicating differential chain length/sulfation for the glycosaminoglycans. We also observed a peak at a retention time of 9.5 minutes that was composed of less than 10% of the total radioactivity. However, in the case of O-linked pyridyl-alpha-xyloside, this early peak accounted for 70% of the total radioactivity observed. The HPLC analyses do not give any information about the composition of the HS/CS in the total glycosaminoglycans. The HPLC analysis of various xylosides is shown in FIGS. 10A-10AE, where the structure corresponding to the HPLC analysis is shown.

### Example 11

A library of triazolyl xylosides were synthesized using the “click” chemistry as described and shown in FIG. 7A-7G. In this regard, we have synthesized various xylosides (Tables 1A-1B) and examined their ability to stimulate the production of GAGs.

A mutant pgsA-745 cell line, which lacks active xylosyltransferase enzyme, does not make GAG chains. It requires exogenous supply of β-xylosides to produce GAGs, and thus makes it a convenient cellular system for determining the priming ability of exogenously supplied Click xylosides (i.e., prepared by Click Chemistry). Previous studies suggest that α-D-xylosides can not prime glycosaminoglycans and in fact early experiments with β-xylosides often used α-xyloside as a negative control. Both β- and α-click xylosides can potentially stimulate the biosynthesis of GAG chains because these click xylosides carry an unusual triazole ring at the anomeric carbon. Therefore, at the outset, a series of β- and α-D-xylosides were synthesized and fed to the mutant CHO cells (pgsA-745). Surprisingly, all of α-D-xylosides (Table 1A: compounds 2, 6, 8, 10 and 23) prime GAGs albeit in lesser quantities than their respective β-anomers (Table 1A: compounds 1, 5, 7, 9 and 22). We have shown that xylosides with a configuration could indeed stimulate GAG biosynthesis (FIG. 2). We synthesized and examined extensively β-xylosides with various aglycone moieties attached through click chemistry. Simple triazole linked β-xyloside (Table 1A: compound 1) primed slightly better than the well known β-O-naphthyl xyloside.

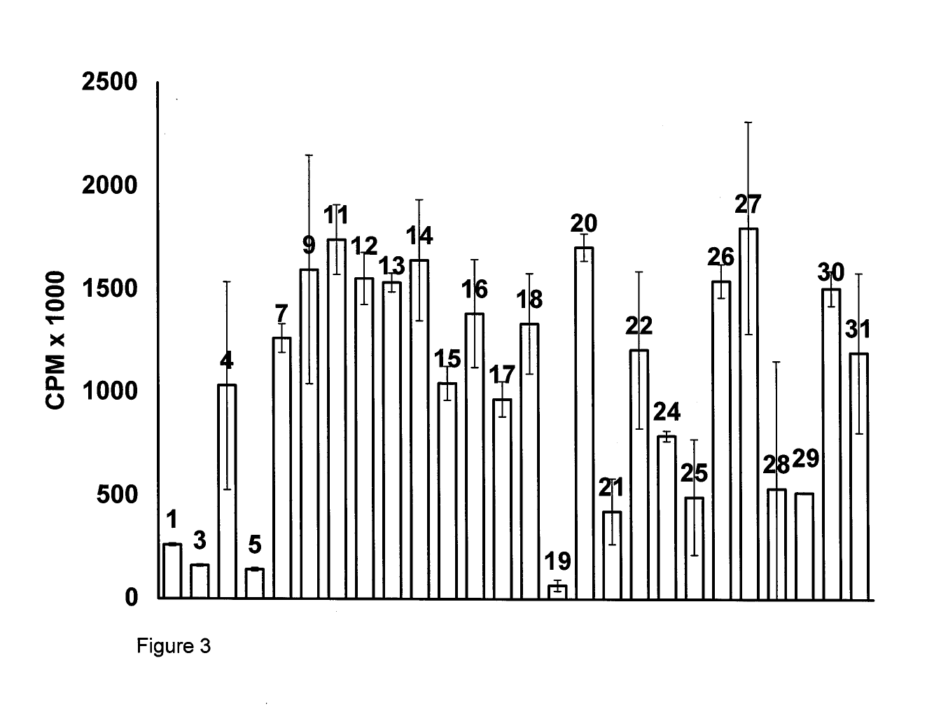

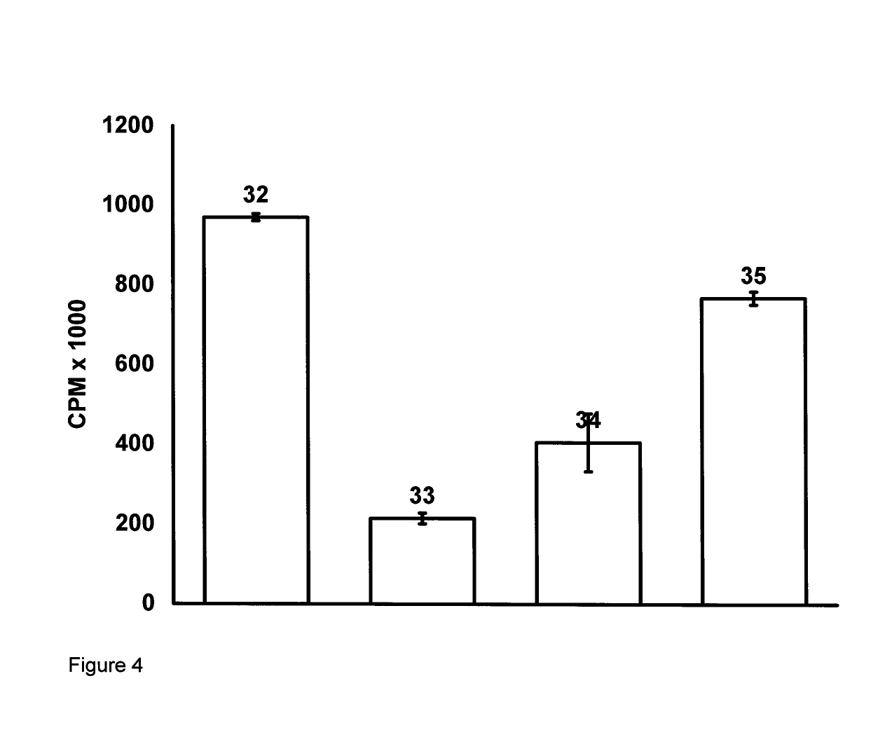

We synthesized click xylosides in which cyclohexyl (Table 1A: compound 4) or phenyl ring attached to the xylose residue via triazole group (Table 1A: compound 7). Both Table 1A compound 4 and 7 primed nearly 4-5 times more GAG chains compared to the simple prototype Table 1A compound 1. This demonstrated the importance of hydrophobic group which confers priming activity to the click xylosides (FIG. 3).

Since the cyclohexyl moiety is less amenable for functional group introduction, we have chosen to utilize aryl moiety containing click xylosides to probe the effect of the various functional groups and the resulting divergent structures for their ability to modulate GAG biosynthesis. Heteroatom substitution in the phenyl ring such as pyridyl group was compared to the phenyl ring in terms of its ability to synthesize the GAG chains. Pyridyl substituted xyloside (Table 1A: compound 5) primed 10 times less efficiently than phenyl substituted xyloside (Table 1A: compound 7). This is in accordance with the earlier observation that heteroatom substitution prohibits the efficient priming of GAG chains.

To test the effect of phenyl ring substitution at para position on the priming ability, a set of xylosides were synthesized with the following substitutions: —OCH3, —F, —Cl, —Br, —I, —NO2 (Table 1A: compound 9, 11-15 respectively). Experimental data suggests that any substitution at para position of phenyl ring has a marginal effect on the priming ability of xylosides except the nitro group which abolished the activity by approximately 30%.

We have scrutinized the effect of polyaromatic substitutions such as biphenyl and naphthyl rings on the priming ability of xylosides. In general, naphthyl and biphenyl moieties are conducive for priming GAG chains. However, it is interesting to note that addition of a Br atom to either biphenyl (Table 1A: compound 17) or naphthyl (Table 1A: compound 19) moiety decreased or completely abolished the priming activity respectively. On the other hand, saturation of one of the naphthyl aromatic ring (Table 1A: compound 20) did not significantly influence priming activity. One xyloside (Table 1A: compound 21) is more hydrophobic than another xyloside (Table 1A: compound 18) and, therefore, the more hydrophobic xyloside can be transported to Golgi more easily, and thus, was predicted to have a higher priming activity. To our surprise, the more hydrophobic xyloside (Table 1A: compound 21) primed less GAG chains than the less hydrophobic xyloside (Table 1A: compound 18) did.

Additionally, the effect of the spacer length between xylose and the hydrophobic aglycone was examined. Removal of —OCH2- spacer (Table 1A: compound 29) decreased the priming ability by two fold. When a methylene group was introduced between the phenyl and triazole, which increases the flexibility of the phenyl ring, the priming ability was same as that of the compound 7 (Table 1A). Similarly, when we replaced phenyl (Table 1A: compound 7) with benzyl group (Table 1A: compound 22), priming ability of the compound 22 was not influenced. However, the introduction of F atom (Table 1A: compound 24) or CH2OH group (Table 1A: compound 25) on the benzyl ring down regulates the priming ability of xylosides. Removal of the OCH2 spacer between naphthyl and the triazole (Table 1A: compound 31 and 18), surprisingly, did not influence the priming activity. These studies clearly indicate that hydrogen bonding interaction between the xyloside and the GAG biosynthetic machinery and the spacer flexibility are essential components for optimal priming.

FIG. 4 illustrates the effectiveness of the xylosides in Table 1B in priming the production of GAGs. Xyloside 32 (Table 1B: compound 32) with ortho substitution primed nearly two times the amount of GAG chains compared to the unsubstituted xyloside 29 (Table 1B: compound 29). On the other hand, the para substituted xyloside 33 (Table 1B: compound 33) primed one-half the amount of GAG chains compared to the unsubstituted xyloside 29 (Table 1B: compound 29) and the meta disubstituted xyloside 34 (Table 1B: compound 34) showed decrease priming compared to the unsubstituted xyloside 29 (Table 1B: compound 29). Thus, the position of substitution has a dramatic influence on the priming ability of the given xyloside.

In conclusion, a library of triazole-linked xylosides with extensive aglycone variations was assembled and examined for their GAG priming activity. We have found that certain modifications are more permissive while others are less permissive for the stimulation of GAG biosynthesis (FIG. 3). These molecules predictably have a longer in vivo half-life, which is likely to influence the biological actions of glycosaminoglycans at much greater level in animal models. Future studies involve characterizing the effect of synthetic xylosides on the production of specific GAG chains and on the sulfation pattern. The stimulated synthesis of core protein free glycosaminoglycan side chains compete with the endogenous proteoglycans for binding to protein ligands at the cell surface and modulate cell behavior. Therefore, these primers are predicted to decipher the biological roles of glycosaminoglycans chains in various model organisms.

TABLE 1A

| entry | R |
|---|---|
| 1 2 | 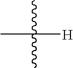 |
| 3 | 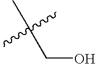 |
| 4 | 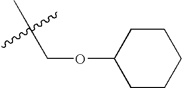 |
| 5 6 | 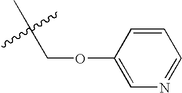 |
| 7 8 | 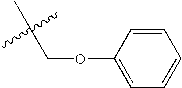 |
| 9 10 | 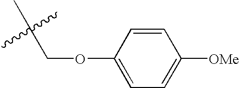 |
| 11 | 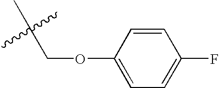 |
| 12 | 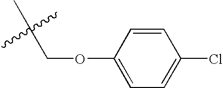 |
| 13 | 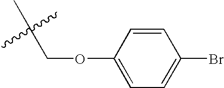 |
| 14 | 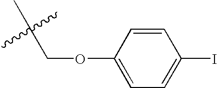 |
| 15 | 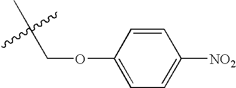 |
| 16 | 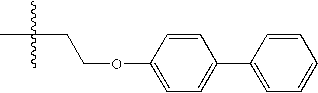 |
| 17 | 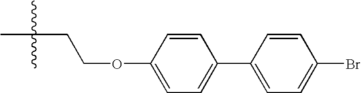 |
| 18 | 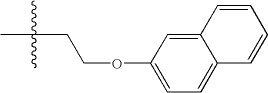 |
| 19 | 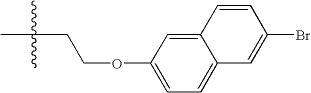 |
| 20 | 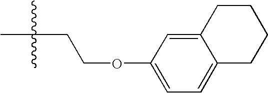 |
| 21 | 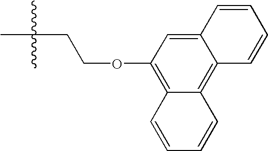 |
| 22 23 | 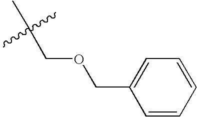 |
| 24 | 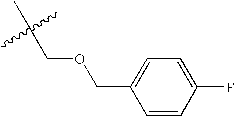 |
| 25 | 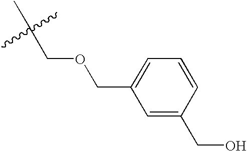 |
| 26 | 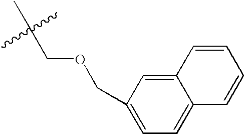 |
| 27 | 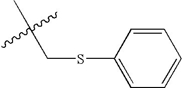 |
| 28 | 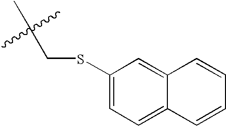 |
| 29 | 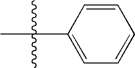 |
| 30 | 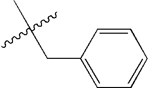 |
| 31 | 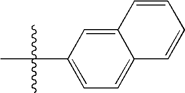 |

TABLE 1B

| Entry | Compound |
|---|---|
| 32 | 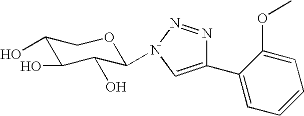 |
| 33 | 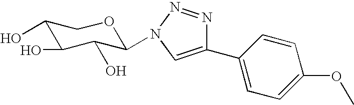 |
| 34 | 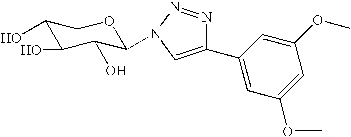 |
| 35 | 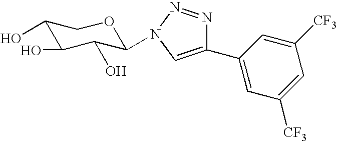 |

TABLE 2

| 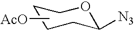 | 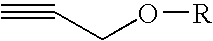 | 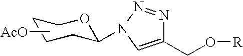 |
|---|---|---|
| 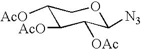 | 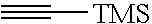 | 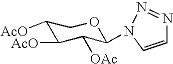 |
| | 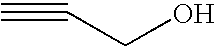 | 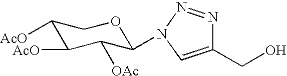 |
| | 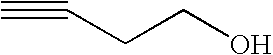 | 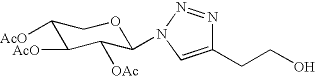 |
| | 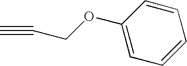 | 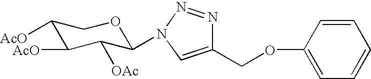 |
| | 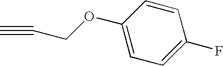 | 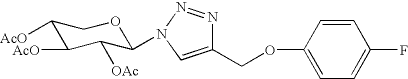 |
| | 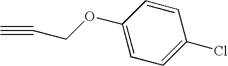 | 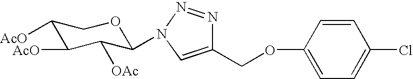 |
| | 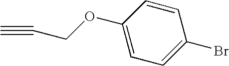 | 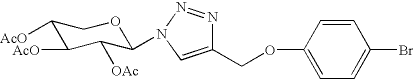 |
| | 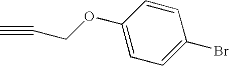 | 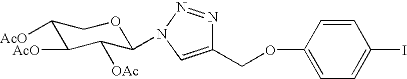 |
| | 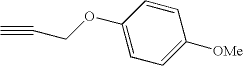 | 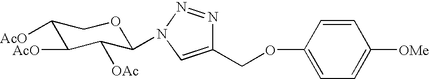 |
| | 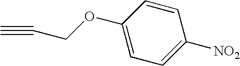 | 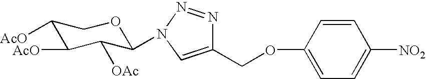 |
| | 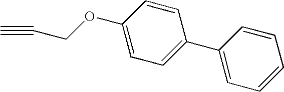 | 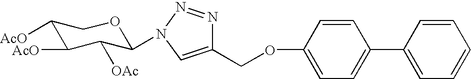 |
| | 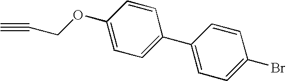 | 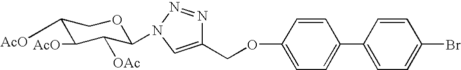 |
| | 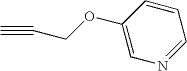 | 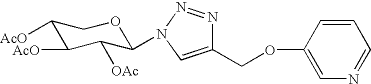 |
| | 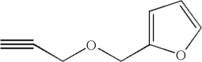 | 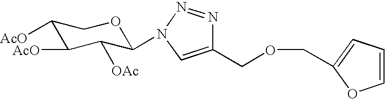 |
| | 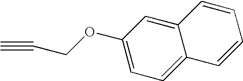 | 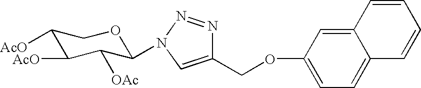 |
| | 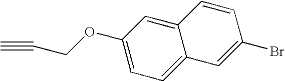 | 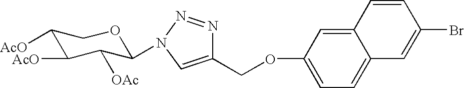 |
| | 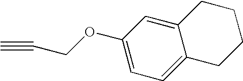 | 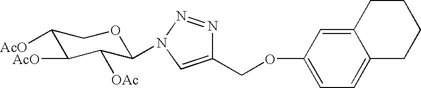 |
| | 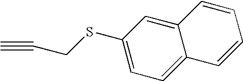 | 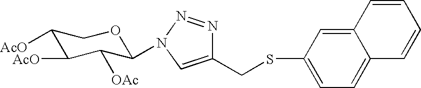 |
| | 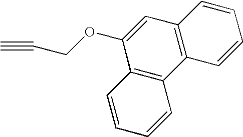 | 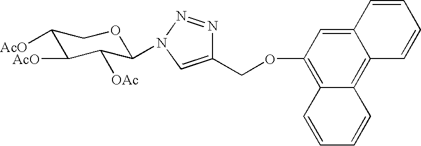 |
| | 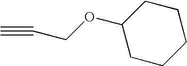 | 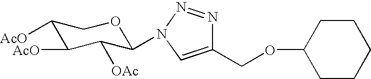 |

TABLE 3

| 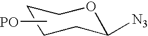 | 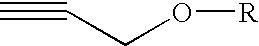 | 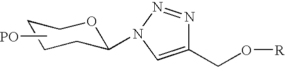 |
|---|---|---|
| 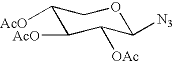 | 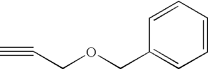 | 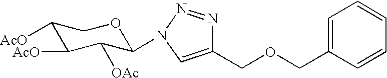 |
| | 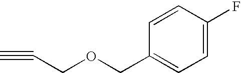 | 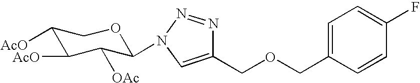 |
| | 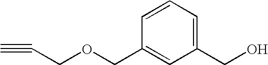 | 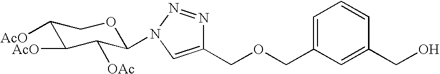 |
| | 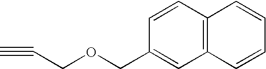 | 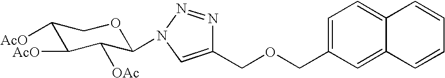 |
| | 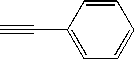 | 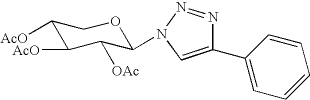 |
| | 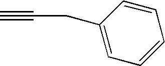 | 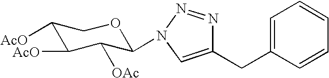 |
| 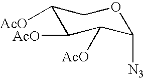 | 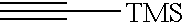 | 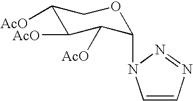 |
| | 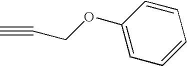 | 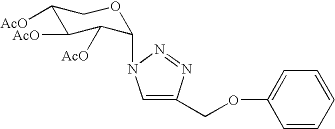 |
| | 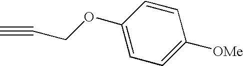 | 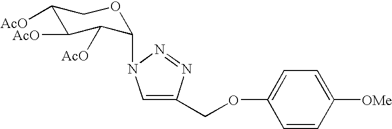 |
| | 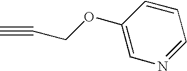 | 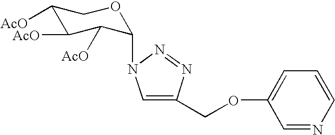 |
| | 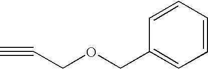 | 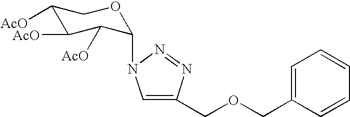 |

TABLE 4

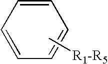

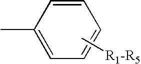

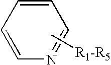

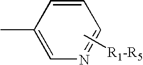

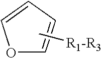

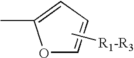

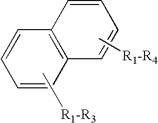

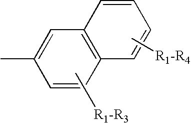

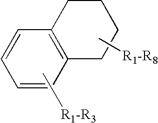

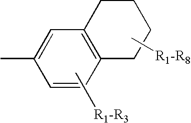

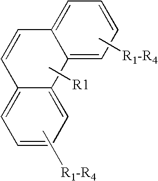

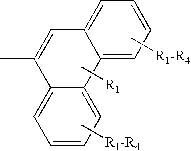

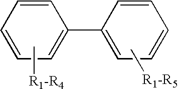

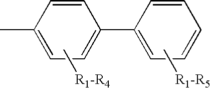

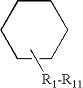

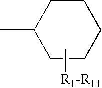

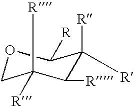

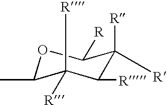

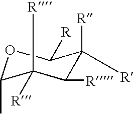

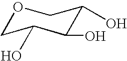

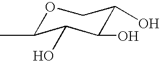

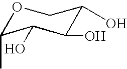

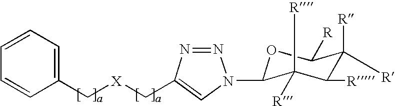

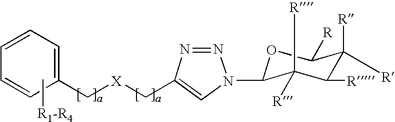

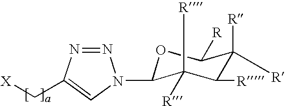

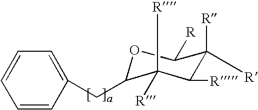

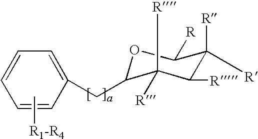

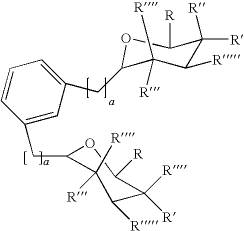

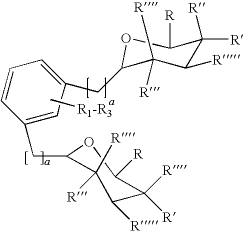

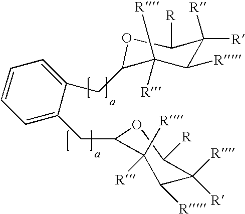

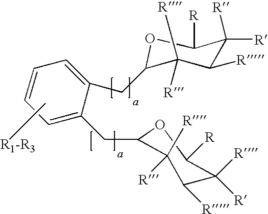

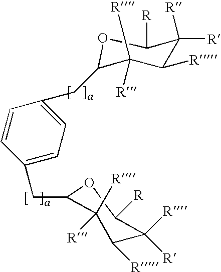

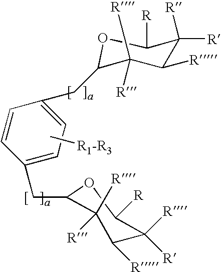

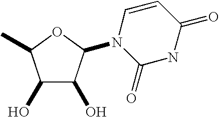

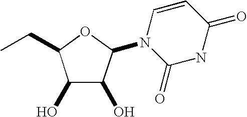

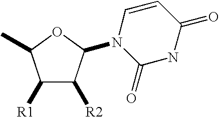

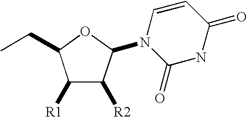

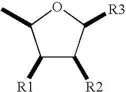

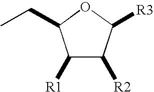

TABLE 5

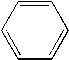

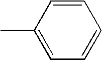

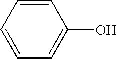

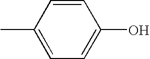

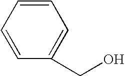

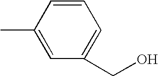

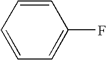

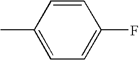

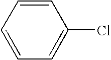

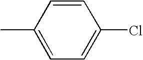

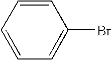

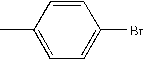

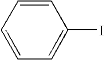

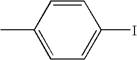

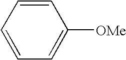

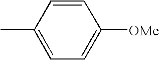

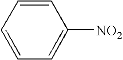

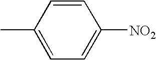

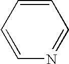

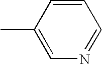

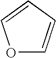

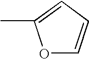

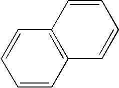

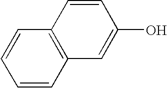

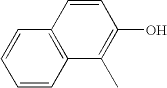

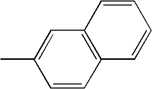

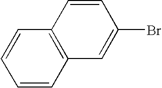

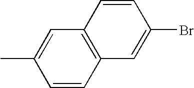

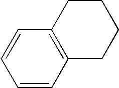

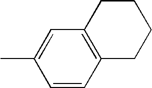

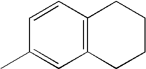

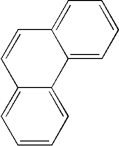

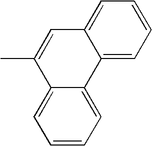

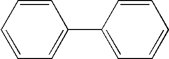

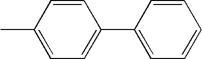

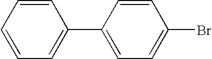

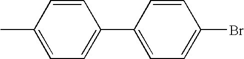

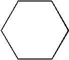

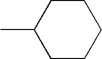

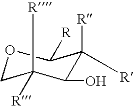

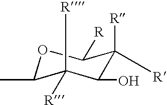

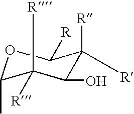

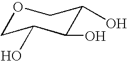

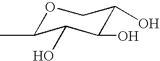

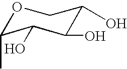

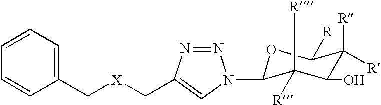

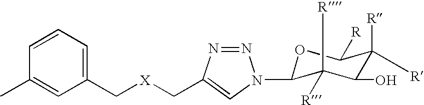

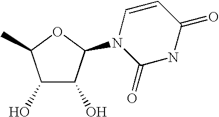

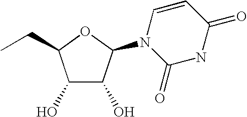

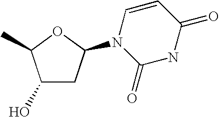

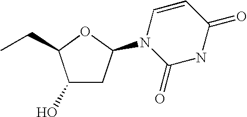

### REFERENCES

[1] J. D. Esko, S. B. Selleck, Annu. Rev. Biochem. 2002, 71, 435.
[2] M. Salmivirta, K. Lidholt, U. Lindahl, FASEB J. 1996, 10, 1270.
[3] A. K. Powell, E. A. Yates, D. G. Fernig, J. E. Turnbull, Glycobiology 2004, 14, 17R.
[4] R. Sasisekharan, Z. Shriver, G. Venkataraman, U. Narayanasami, Nat. Rev. Cancer. 2002, 2, 521.
[5] A. D. Lander, Curr. Opin. Neurobiol. 1993, 3, 716.
[6] I. Capila, R. J. Linhardt, Angew. Chem. Int. Ed. Engl. 2002, 41, 391.
[7] N. B. Schwartz, L. Galligani, P. L. Ho, A. Dorfman, Proc. Natl. Acad. Sci. U.S.A. 1974, 71, 4047.
[8] M. Okayama, K. Kimata, S. Suzuki, J. Biochem (Tokyo). 1973, 74, 1069.
[9] M. Sobue, H. Habuchi, K. Ito, H. Yonekura, K. Oguri, K. Sakurai, S. Kamohara, Y. Ueno, R. Noyori, S. Suzuki, Biochem. J. 1987, 241, 591.
[10] T. A. Fritz, F. N. Lugemwa, A. K. Sarkar, J. D. Esko, J. Biol. Chem. 1994, 269, 300.
[11] F. N. Lugemwa, A. K. Sarkar, J. D. Esko, J. Biol. Chem. 1996, 271, 19159.
[12] K. Mani, M. Belting, U. Ellervik, N. Falk, G. Svensson, S. Sandgren, F. Cheng, L. A. Fransson, Glycobiology 2004, 14, 387.
[13] J. L. Platt, D. M. Brown, K. Granlund, T. R. Oegema, D. J. Klein, Dev. Biol. 1987, 123, 293.
[14]H. J. Yost, Development 1990, 110, 865.
[15] A. Rapraeger, J. Cell. Biol. 1989, 109, 2509.
[16] J. Malmberg, K. Mani, E. Sawen, A. Wiren, U. Ellervik, Bioorg. Med. Chem. 2006, 14, 6659.
[17] H. Kolb, M. Finn, K. Sharpless, Angew. Chem. Int. Ed Engl. 2001, 40, 2004.
[18] L. Rossi, A. Basu, Bioorg. Med. Chem. Lett. 2005, 15, 3596.
[19] J. D. Esko, T. E. Stewart, W. H. Taylor, Proc. Natl. Acad. Sci. U. S. A. 1985, 82, 3197.
[20]H. C. Robinson, M. J. Brett, P. J. Tralaggan, D. A. Lowther, M. Okayama, Biochem. J. 1975, 148, 25.
[21]H. H. Freeze, D. Sampath, A. Varki, J. Biol. Chem. 1993, 268, 1618.

1. A xyloside for use in inducing synthesis of a glycosaminoglycan in a cell, the xyloside comprising:
a chemical structure of one of Formula 1, Formula 2, Formula 3, Formula 4, Formula 5, Formula 6, Formula 7, Formula 8, Formula 9, or Formula 10; 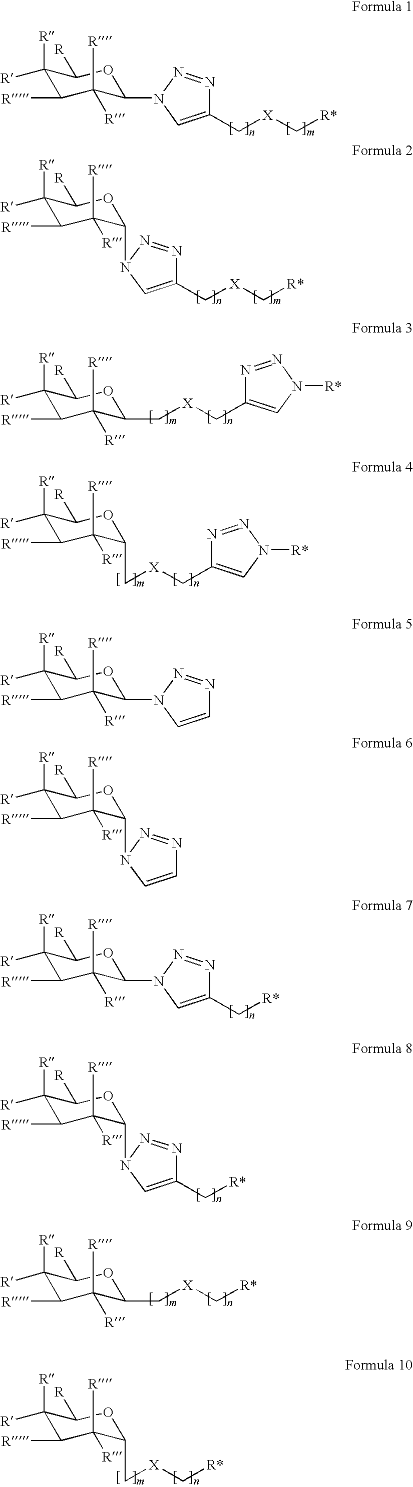
n is from 0 to 10;
m is from 0 to 10;
X is one of a bond, S, SO₂, O, N, or C;
R is one of H, CH₂OH, halogen, COOH, acetyl-oxy, monosaccharide, disaccharide, oligosaccharide, or OH;
R′ is one of H, CH₂OH, halogen, COOH, acetyl-oxy, monosaccharide, disaccharide, oligosaccharide, or OH;
R″ is one of H, CH₂OH, halogen, COOH, acetyl-oxy, monosaccharide, disaccharide, oligosaccharide, or OH;
R″′ is one of H, CH₂OH, halogen, COOH, acetyl-oxy, monosaccharide, disaccharide, oligosaccharide, or OH;
R″″ is one of H, CH₂OH, halogen, COOH, acetyl-oxy, monosaccharide, disaccharide, oligosaccharide, or OH;
R″″′ is one of H, CH₂OH, halogen, COOH, acetyl-oxy, monosaccharide, disaccharide, oligosaccharide, or OH; and
R* is selected from the group consisting of H, OH, CH₂OH, an unsubstituted aliphatic group, a substituted aliphatic group, a halo substituted aliphatic group, a straight chain aliphatic group, a branched chain aliphatic group, alkoxy, a cyclic aliphatic group, an aliphatic group having at least one hetero chain atom, a cyclic aliphatic group having at least one hetero ring atom, an unsubstituted aromatic group, a substituted aromatic group, a halo substituted aromatic group, a polyaromatic group, a substituted polyaromatic group, an aromatic group having at least one hetero ring atom, a polyaromatic group having at least one hetero ring atom, a nucleoside, a nucleotide, a carbohydrate, monosaccharide, disaccharide, oligosaccharide, a polysaccharide; an amino acid, a polypeptide, combinations thereof, and derivatives thereof.

2. A xyloside for use in inducing synthesis of a glycosaminoglycan in a cell, the xyloside comprising:
a chemical structure of one of Formula 1, Formula 2, Formula 3, or Formula 4; 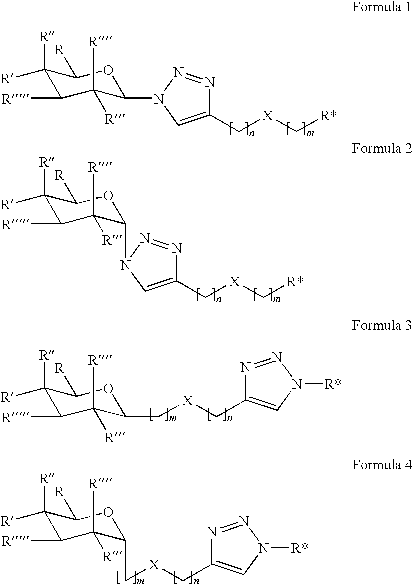
n is from 0 to 10;
m is from 0 to 10;
X is one of S, O, N, or C;
R is one of H, CH₂OH, halogen, COOH, acetyl-oxy, monosaccharide, disaccharide, oligosaccharide, or OH;
R′ is one of H, CH₂OH, halogen, COOH, acetyl-oxy, monosaccharide, disaccharide, oligosaccharide, or OH;
R″ is one of H, CH₂OH, halogen, COOH, acetyl-oxy, monosaccharide, disaccharide, oligosaccharide, or OH;
R″′ is one of H, CH₂OH, halogen, COOH, acetyl-oxy, monosaccharide, disaccharide, oligosaccharide, or OH;
R″″ is one of H, CH₂OH, halogen, COOH, acetyl-oxy, monosaccharide, disaccharide, oligosaccharide, or OH;
R″″′ is one of H, CH₂OH, halogen, COOH, acetyl-oxy, monosaccharide, disaccharide, oligosaccharide, or OH; and
R* is selected from the group consisting of H, OH, CH₂OH, an unsubstituted aliphatic group, a substituted aliphatic group, a halo substituted aliphatic group, a straight chain aliphatic group, a branched chain aliphatic group, alkoxy, a cyclic aliphatic group, an aliphatic group having at least one hetero chain atom, a cyclic aliphatic group having at least one hetero ring atom, an unsubstituted aromatic group, a substituted aromatic group, a halo substituted aromatic group, a polyaromatic group, a substituted polyaromatic group, an aromatic group having at least one hetero ring atom, a polyaromatic group having at least one hetero ring atom, a nucleoside, a nucleotide, a carbohydrate, monosaccharide, disaccharide, oligosaccharide, a polysaccharide; an amino acid, a polypeptide, combinations thereof, and derivatives thereof.

3. A xyloside for use in inducing synthesis of a glycosaminoglycan in a cell, the xyloside comprising:
a chemical structure of one of Formula 5 or Formula 6; 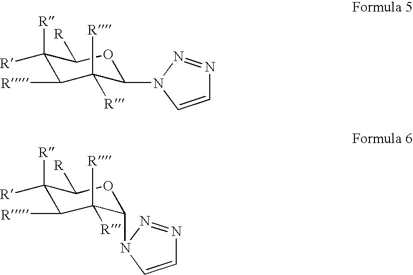
R is one of H, CH₂OH, halogen, COOH, acetyl-oxy, monosaccharide, disaccharide, oligosaccharide, or OH;
R′ is one of H, CH₂OH, halogen, COOH, acetyl-oxy, monosaccharide, disaccharide, oligosaccharide, or OH;
R″ is one of H, CH₂OH, halogen, COOH, acetyl-oxy, monosaccharide, disaccharide, oligosaccharide, or OH;
R″′ is one of H, CH₂OH, halogen, COOH, acetyl-oxy, monosaccharide, disaccharide, oligosaccharide, or OH;
R″″ is one of H, CH₂OH, halogen, COOH, acetyl-oxy, monosaccharide, disaccharide, oligosaccharide, or OH; and
R″″′ is one of H, CH₂OH, halogen, COOH, acetyl-oxy, monosaccharide, disaccharide, oligosaccharide, or OH.

4. A xyloside for use in inducing synthesis of a glycosaminoglycan in a cell, the xyloside comprising:
a chemical structure of one of Formula 7 or Formula 8; 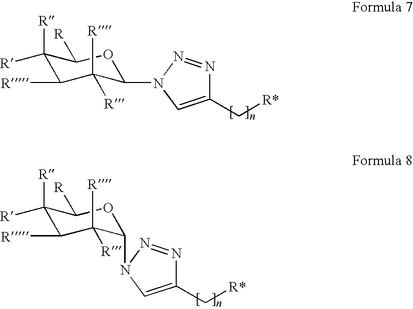
n is from 0 to 10;
R is one of H, CH₂OH, halogen, COOH, acetyl-oxy, monosaccharide, disaccharide, oligosaccharide, or OH;
R′ is one of H, CH₂OH, halogen, COOH, acetyl-oxy, monosaccharide, disaccharide, oligosaccharide, or OH;
R″ is one of H, CH₂OH, halogen, COOH, acetyl-oxy, monosaccharide, disaccharide, oligosaccharide, or OH;
R″′ is one of H, CH₂OH, halogen, COOH, acetyl-oxy, monosaccharide, disaccharide, oligosaccharide, or OH;
R″″ is one of H, CH₂OH, halogen, COOH, acetyl-oxy, monosaccharide, disaccharide, oligosaccharide, or OH;
R″″′ is one of H, CH₂OH, halogen, COOH, acetyl-oxy, monosaccharide, disaccharide, oligosaccharide, or OH; and
R* is selected from the group consisting of H, OH, CH₂OH, an unsubstituted aliphatic group, a substituted aliphatic group, a halo substituted aliphatic group, a straight chain aliphatic group, a branched chain aliphatic group, alkoxy, a cyclic aliphatic group, an aliphatic group having at least one hetero chain atom, a cyclic aliphatic group having at least one hetero ring atom, an unsubstituted aromatic group, a substituted aromatic group, a halo substituted aromatic group, a polyaromatic group, a substituted polyaromatic group, an aromatic group having at least one hetero ring atom, a polyaromatic group having at least one hetero ring atom, a nucleoside, a nucleotide, a carbohydrate, monosaccharide, disaccharide, oligosaccharide, a polysaccharide; an amino acid, a polypeptide, combinations thereof, and derivatives thereof.

5. A xyloside for use in inducing synthesis of a glycosaminoglycan in a cell, the xyloside comprising:
a chemical structure of one of Formula 9 or Formula 10; 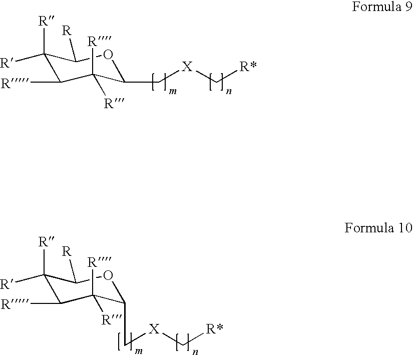
n is from 0 to 10;
m is from 0 to 10;
X is one of a bond, S, SO₂, O, N, or C;
R is one of H, CH₂OH, halogen, COOH, acetyl-oxy, monosaccharide, disaccharide, oligosaccharide, or OH;
R′ is one of H, CH₂OH, halogen, COOH, acetyl-oxy, monosaccharide, disaccharide, oligosaccharide, or OH;
R″ is one of H, CH₂OH, halogen, COOH, acetyl-oxy, monosaccharide, disaccharide, oligosaccharide, or OH;
R′″ is one of H, CH₂OH, halogen, COOH, acetyl-oxy, monosaccharide, disaccharide, oligosaccharide, or OH;
R″″ is one of H, CH₂OH, halogen, COOH, acetyl-oxy, monosaccharide, disaccharide, oligosaccharide, or OH;
R″″′ is one of H, CH₂OH, halogen, COOH, acetyl-oxy, monosaccharide, disaccharide, oligosaccharide, or OH; and
R* is selected from the group consisting of H, OH, CH₂OH, an unsubstituted aliphatic group, a substituted aliphatic group, a halo substituted aliphatic group, a straight chain aliphatic group, a branched chain aliphatic group, alkoxy, a cyclic aliphatic group, an aliphatic group having at least one hetero chain atom, a cyclic aliphatic group having at least one hetero ring atom, an unsubstituted aromatic group, a substituted aromatic group, a halo substituted aromatic group, a polyaromatic group, a substituted polyaromatic group, an aromatic group having at least one hetero ring atom, a polyaromatic group having at least one hetero ring atom, a nucleoside, a nucleotide, a carbohydrate, monosaccharide, disaccharide, oligosaccharide, a polysaccharide; an amino acid, a polypeptide, combinations thereof, and derivatives thereof.

6. A method of making a xyloside for use in inducing synthesis of a glycosaminoglycan in a cell, the method comprising:
obtaining an N₃-containing reagent having a chemical structure as in one of Reagent 1A, Reagent 1B, Reagent 1C, or Reagent 1D, wherein at least one of R, R′, R″, R″′, R″″, or R″″′ is protected by a protecting group that is capable of being deprotected without degrading the xyloside; 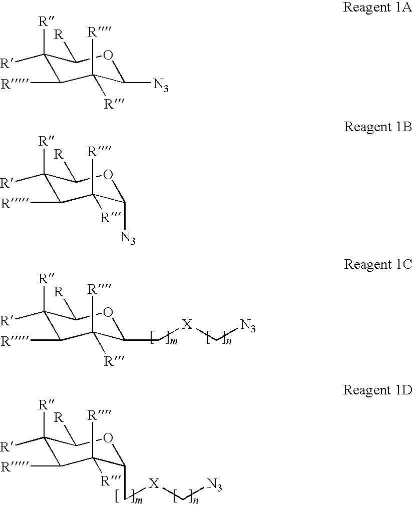
obtaining an alkynyl-containing reagent having a chemical structure as in one of Reagent 2A, Reagent 2B, or Reagent 2C; 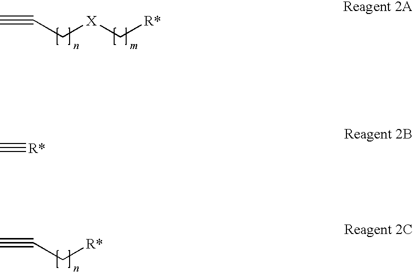
reacting one of Reagents 1A-1D with one of Reagents 2A-2C; and
deprotecting the at least one of R, R′, R″, R″′, R″″, or R″″′ that is protected by the protecting group so as to arrive at the at least one of R, R′, R″, R″′, R″″, or R″″′ so as to obtain the xyloside, wherein
n is from 0 to 10;
m is from 0 to 10;
X is one of a bond, S, SO₂, O, N, or C;
R is one of H, CH₂OH, halogen, COOH, acetyl-oxy, monosaccharide, disaccharide, oligosaccharide, or OH;
R′ is one of H, CH₂OH, halogen, COOH, acetyl-oxy, monosaccharide, disaccharide, oligosaccharide, or OH;
R″ is one of H, CH₂OH, halogen, COOH, acetyl-oxy, monosaccharide, disaccharide, oligosaccharide, or OH;
R″′ is one of H, CH₂OH, halogen, COOH, acetyl-oxy, monosaccharide, disaccharide, oligosaccharide, or OH;
R″″ is one of H, CH₂OH, halogen, COOH, acetyl-oxy, monosaccharide, disaccharide, oligosaccharide, or OH;
R″″′ is one of H, CH₂OH, halogen, COOH, acetyl-oxy, monosaccharide, disaccharide, oligosaccharide, or OH; and
R* is selected from the group consisting of H, OH, CH₂OH, an unsubstituted aliphatic group, a substituted aliphatic group, a halo substituted aliphatic group, a straight chain aliphatic group, a branched chain aliphatic group, alkoxy, a cyclic aliphatic group, an aliphatic group having at least one hetero chain atom, a cyclic aliphatic group having at least one hetero ring atom, an unsubstituted aromatic group, a substituted aromatic group, a halo substituted aromatic group, a polyaromatic group, a substituted polyaromatic group, an aromatic group having at least one hetero ring atom, a polyaromatic group having at least one hetero ring atom, a nucleoside, a nucleotide, a carbohydrate, monosaccharide, disaccharide, oligosaccharide, a polysaccharide; an amino acid, a polypeptide, combinations thereof, and derivatives thereof.

7. A method of making a xyloside for use in inducing synthesis of a glycosaminoglycan in a cell, the method comprising:
obtaining an alkynyl-containing reagent having a chemical structure as in one of Reagent 3A or Reagent 3B, wherein at least one of R, R′, R″, R″′, R″″, or R″″′ is protected by a protecting group that can be deprotected without degrading the xyloside; 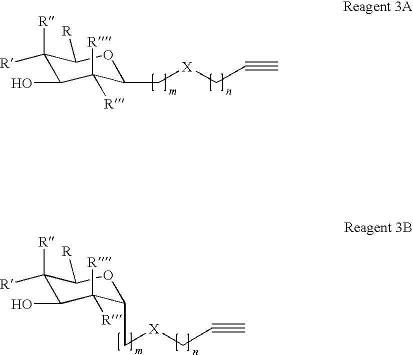
obtaining an N₃-containing reagent having a chemical structure as in Reagent 4A;
N₃—R^{*} Reagent 4A
reacting one of Reagent 3A-3B with Reagent 4A; and
deprotecting the at least one of R, R′, R″, R″′, R″″, or R′″″ that is protected by the protecting group so as to arrive at the at least one of R, R′, R″, R′″, R″″, or R″″′ so as to obtain the xyloside, wherein
n is from 0 to 10;
m is from 0 to 10;
X is one of a bond, S, SO₂, O, N, or C;
R is one of H, CH₂OH, halogen, COOH, acetyl-oxy, monosaccharide, disaccharide, oligosaccharide, or OH;
R′ is one of H, CH₂OH, halogen, COOH, acetyl-oxy, monosaccharide, disaccharide, oligosaccharide, or OH;
R″ is one of H, CH₂OH, halogen, COOH, acetyl-oxy, monosaccharide, disaccharide, oligosaccharide, or OH;
R″′ is one of H, CH₂OH, halogen, COOH, acetyl-oxy, monosaccharide, disaccharide, oligosaccharide, or OH;
R″″ is one of H, CH₂OH, halogen, COOH, acetyl-oxy, monosaccharide, disaccharide, oligosaccharide, or OH;
R″″′ is one of H, CH₂OH, halogen, COOH, acetyl-oxy, monosaccharide, disaccharide, oligosaccharide, or OH; and
R* is selected from the group consisting of H, OH, CH₂OH, an unsubstituted aliphatic group, a substituted aliphatic group, a halo substituted aliphatic group, a straight chain aliphatic group, a branched chain aliphatic group, alkoxy, a cyclic aliphatic group, an aliphatic group having at least one hetero chain atom, a cyclic aliphatic group having at least one hetero ring atom, an unsubstituted aromatic group, a substituted aromatic group, a halo substituted aromatic group, a polyaromatic group, a substituted polyaromatic group, an aromatic group having at least one hetero ring atom, a polyaromatic group having at least one hetero ring atom, a nucleoside, a nucleotide, a carbohydrate, monosaccharide, disaccharide, oligosaccharide, a polysaccharide; an amino acid, a polypeptide, combinations thereof, and derivatives thereof.

8. A method of using a xyloside for inducing synthesis of a glycosaminoglycan in a cell, the method comprising:
providing a xyloside;
introducing the xyloside into the cell; and
maintaining the cell under conditions in which the xyloside is capable of inducing the cell to synthesize the glycosaminoglycan,
wherein the xyloside has a chemical structure of one of Formula 1, Formula 2, Formula 3, Formula 4, Formula 5, Formula 6, Formula 7, Formula 8, Formula 9, or Formula 10; 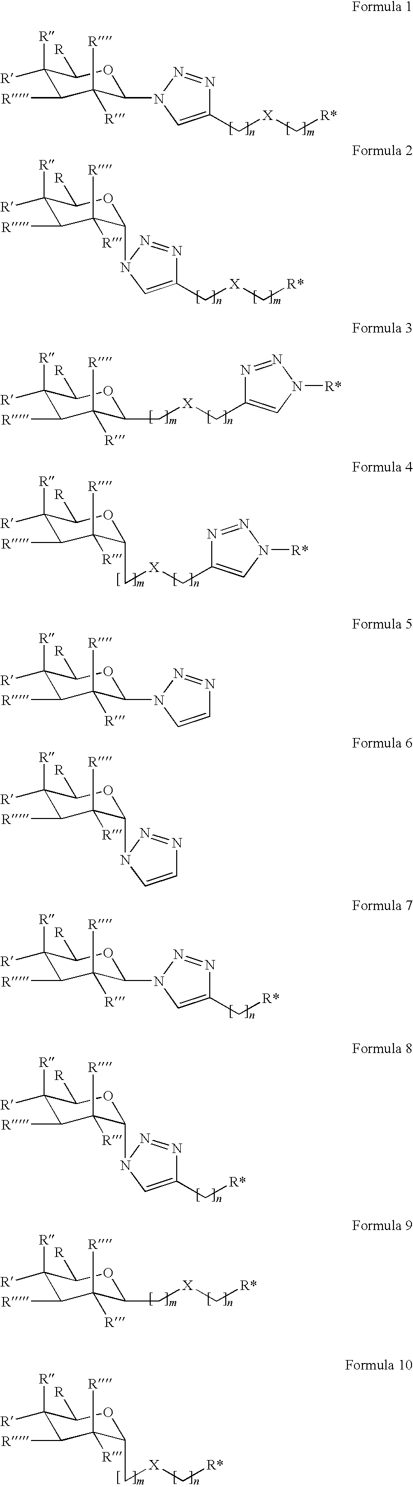
n is from 0 to 10;
m is from 0 to 10;
X is one of a bond, S, SO₂, O, N, or C;
R is one of H, CH₂OH, halogen, COOH, acetyl-oxy, monosaccharide, disaccharide, oligosaccharide, or OH;
R′ is one of H, CH₂OH, halogen, COOH, acetyl-oxy, monosaccharide, disaccharide, oligosaccharide, or OH;
R″ is one of H, CH₂OH, halogen, COOH, acetyl-oxy, monosaccharide, disaccharide, oligosaccharide, or OH;
R″′ is one of H, CH₂OH, halogen, COOH, acetyl-oxy, monosaccharide, disaccharide, oligosaccharide, or OH;
R″″ is one of H, CH₂OH, halogen, COOH, acetyl-oxy, monosaccharide, disaccharide, oligosaccharide, or OH;
R″″′ is one of H, CH₂OH, halogen, COOH, acetyl-oxy, monosaccharide, disaccharide, oligosaccharide, or OH; and
R* is selected from the group consisting of H, OH, CH₂OH, an unsubstituted aliphatic group, a substituted aliphatic group, a halo substituted aliphatic group, a straight chain aliphatic group, a branched chain aliphatic group, alkoxy, a cyclic aliphatic group, an aliphatic group having at least one hetero chain atom, a cyclic aliphatic group having at least one hetero ring atom, an unsubstituted aromatic group, a substituted aromatic group, a halo substituted aromatic group, a polyaromatic group, a substituted polyaromatic group, an aromatic group having at least one hetero ring atom, a polyaromatic group having at least one hetero ring atom, a nucleoside, a nucleotide, a carbohydrate, monosaccharide, disaccharide, oligosaccharide, a polysaccharide; an amino acid, a polypeptide, combinations thereof, and derivatives thereof.

9. A xyloside or method as in one of claims 1-8, wherein R* is one of the following: 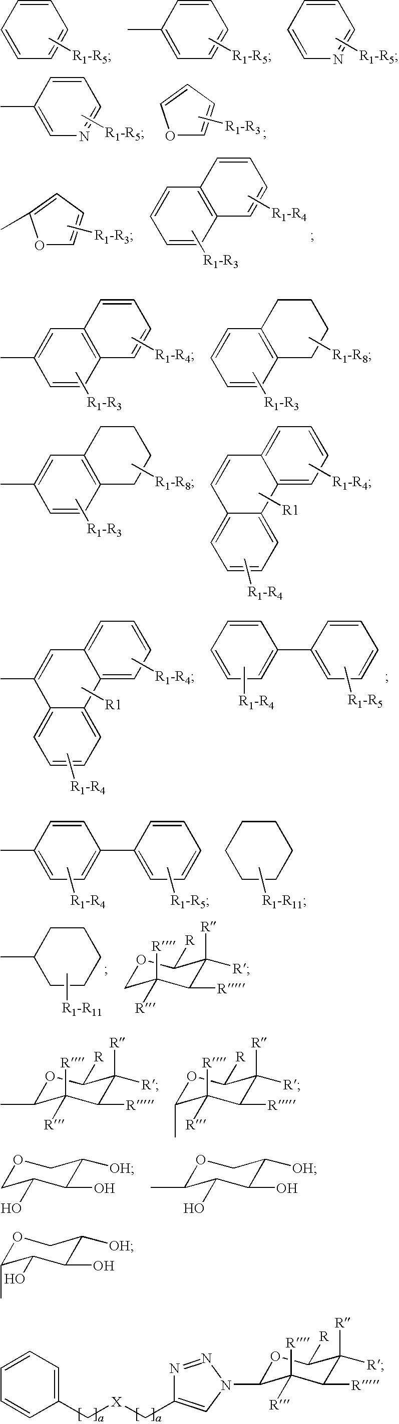 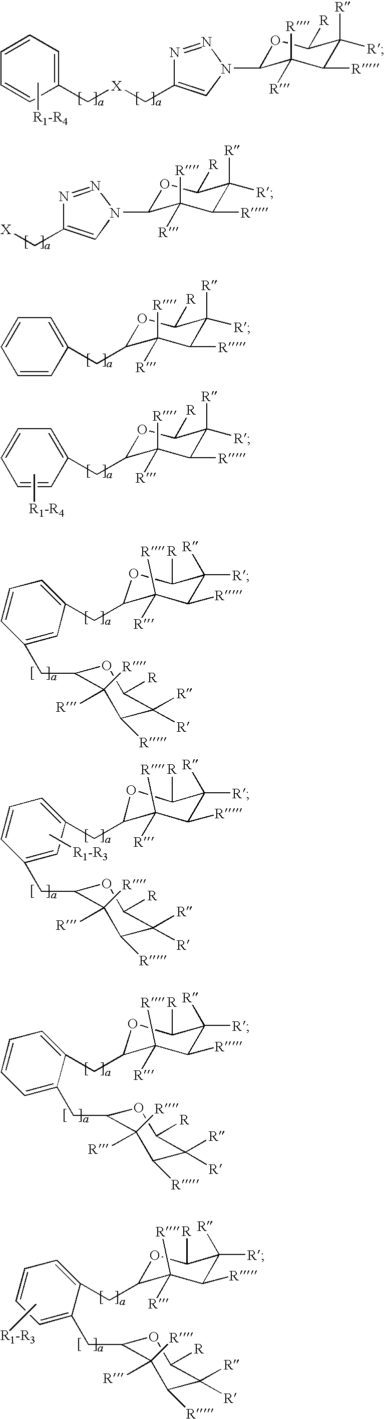 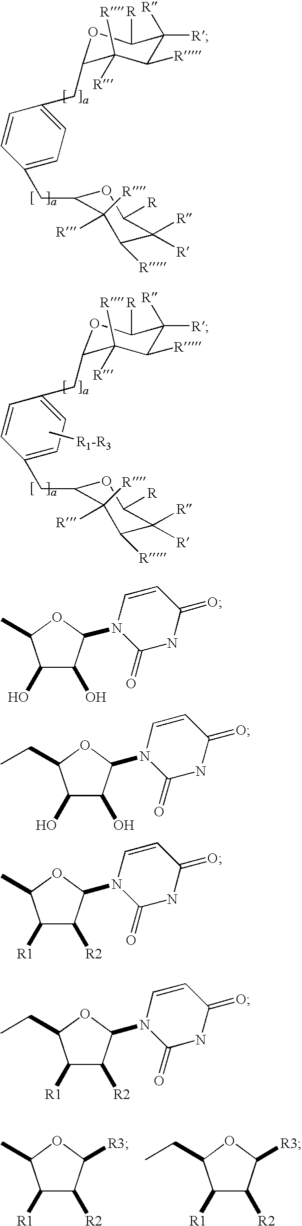
H, OH; CH2OH; combinations thereof, and derivatives thereof;
wherein each of R1-R11 are individually selected from H, OH, CH₂OH, halogen, F, Cl, Br, I, alkoxy, methoxy, NO₂, unsubstituted aliphatic group, a substituted aliphatic group, a halo substituted aliphatic group, a straight chain aliphatic group, a branched chain aliphatic group, a cyclic aliphatic group, an aliphatic group having at least one hetero chain atom, a cyclic aliphatic group having at least one hetero ring atom, an unsubstituted aromatic group, a substituted aromatic group, a halo substituted aromatic group, a polyaromatic group, a substituted polyaromatic group, an aromatic group having at least one hetero ring atom, a polyaromatic group having at least one hetero ring atom, a nucleoside, a nucleotide, a carbohydrate, monosaccharide, disaccharide, oligosaccharide, a polysaccharide; an amino acid, a polypeptide, adenine, guanine, thymine, uracil, cytosine, combinations thereof, and derivatives thereof.

10. A xyloside or method as in one of claims 1-8, wherein R* is one of the following: 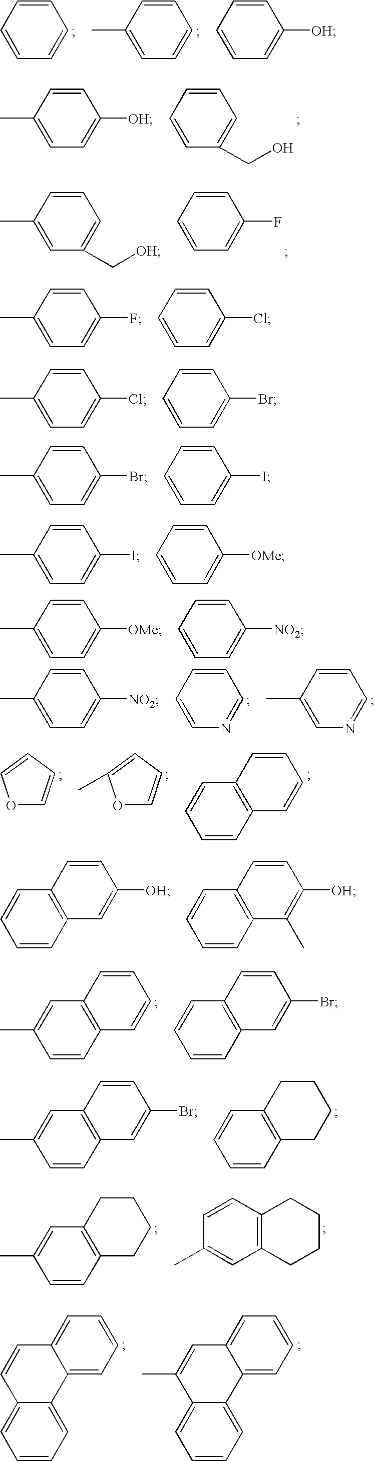 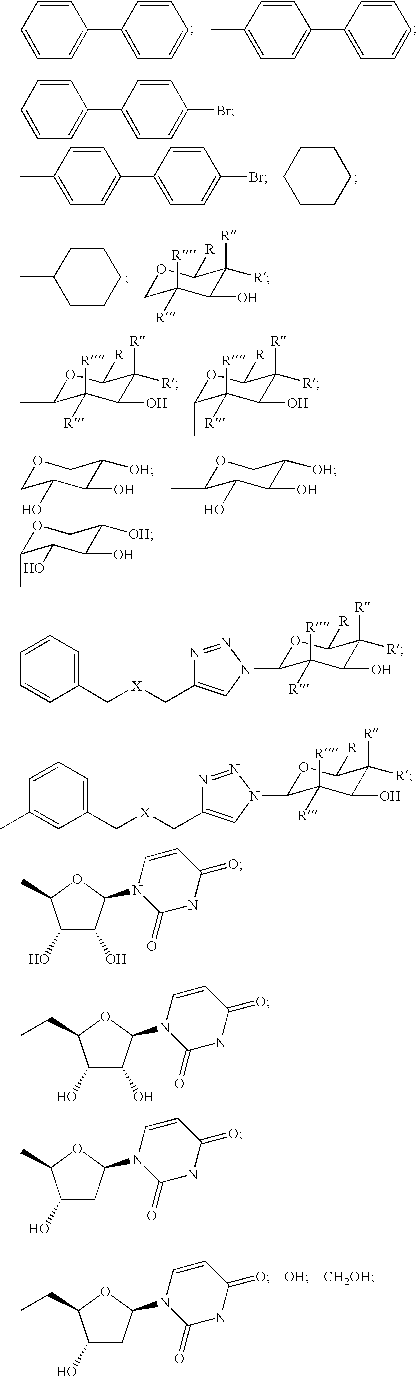
combinations thereof, and derivatives thereof.

11. A xyloside or method as in one of claims 1-8, wherein m is 0 or 1 and/or n is 0 or 1.

12. A xyloside or method as in one of claims 1-8, characterized by at least one of the following:
when R′ is H, then R″ is OH;
when R′ is OH or F, then R″ is H;
when R″′ is OH, then R″″ is H;
when R″′ is H, then R″″ is OH;
at least one of R, R′, R″, R″′, or R″″ is H;
at least one of R, R′, R″, R″′, or R″″ is OH;
at least one of R, R′, R″, R″′, or R″″ is F;
at least one of R is H, R′ is OH, R″ is H, R″′ is OH, or R″″ is H;
the aliphatic group of R* has a main chain of 1 to 10 carbons;
the aromatic group or polyaromatic group of R* has from 1 to 3 aromatic rings.

13. A xyloside or method as in one of claims 1-8, wherein the xyloside is selected from the following xylosides:

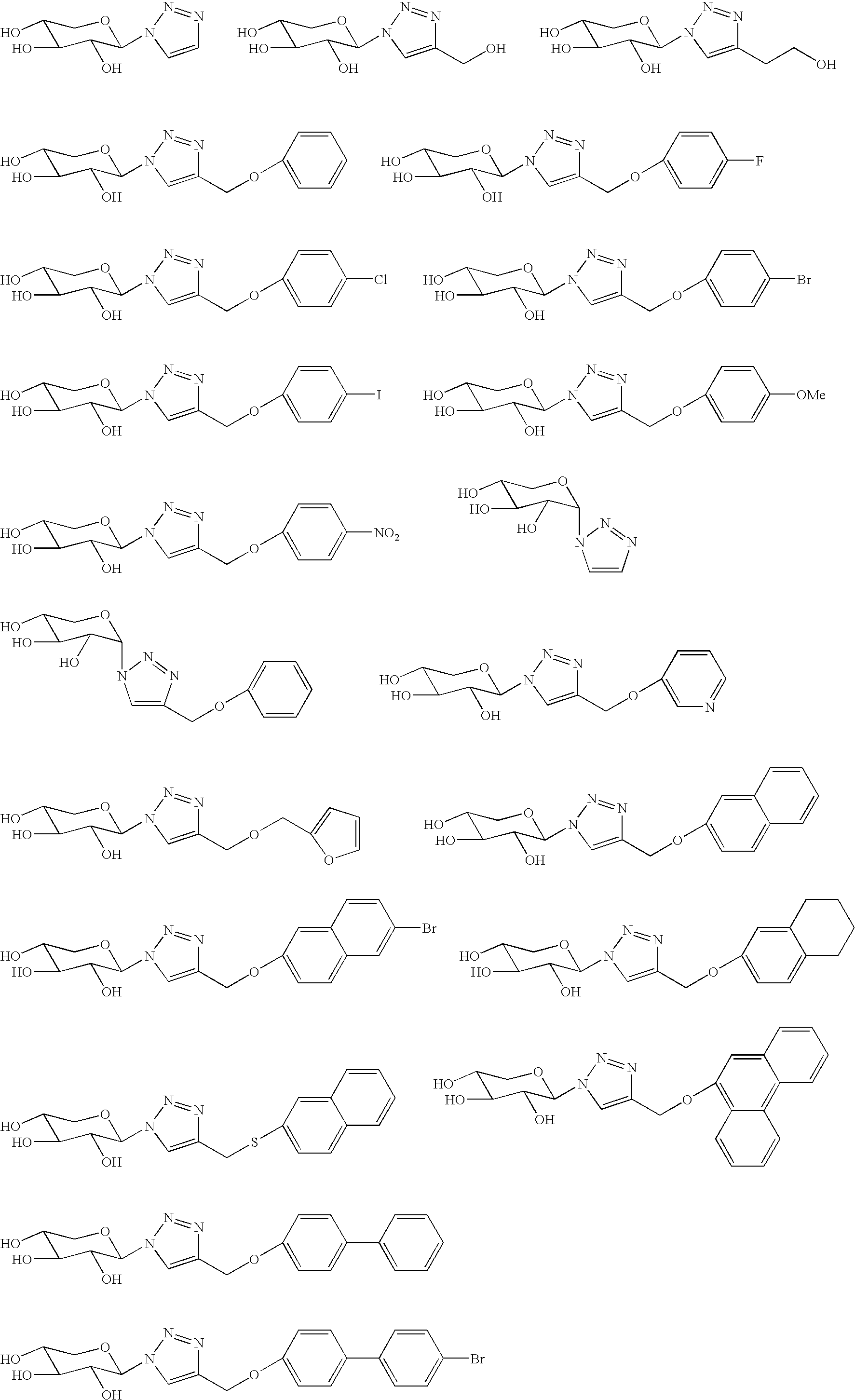

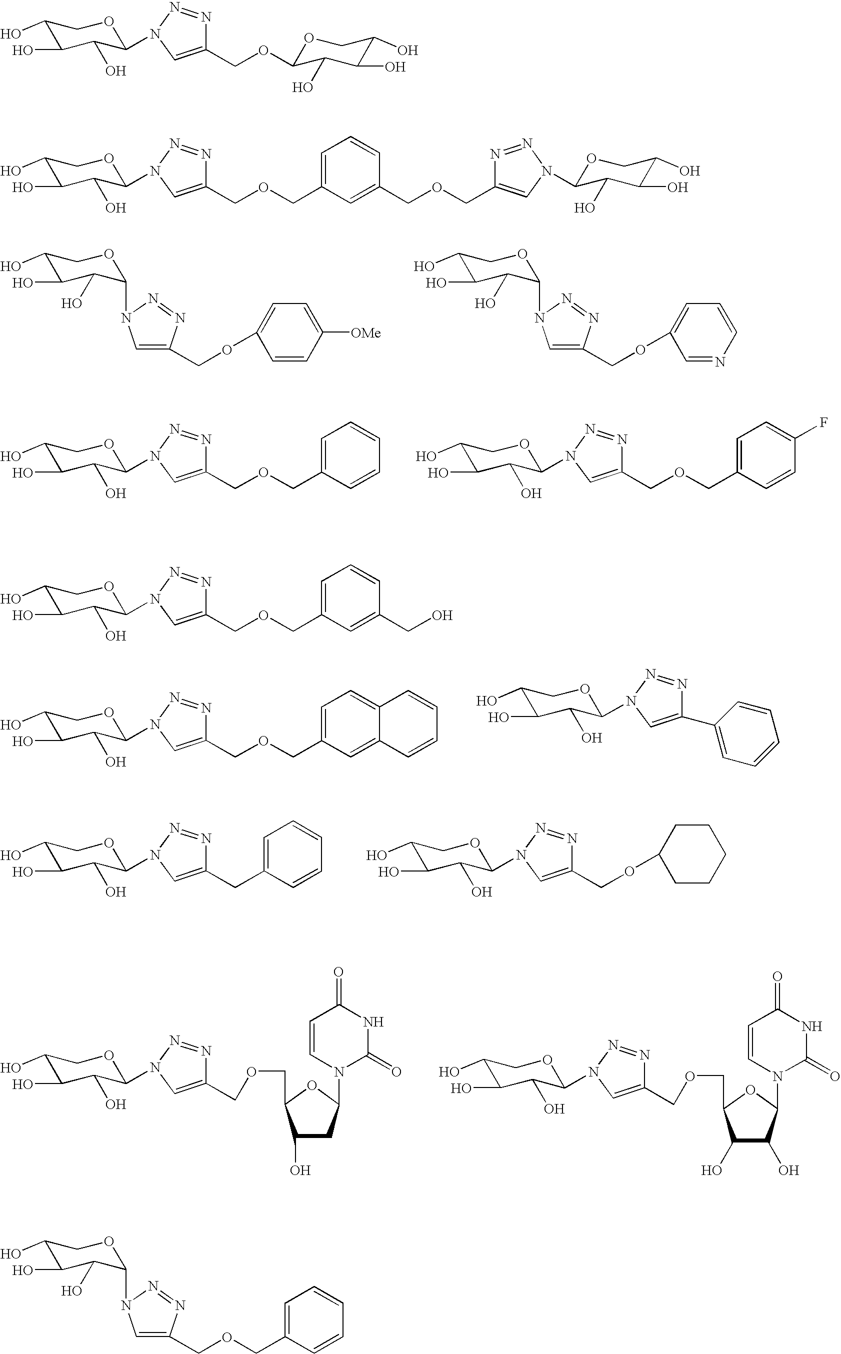

14. A xyloside or method as in one of claims 1-8, wherein the an N₃-containing reagent, alkynyl-containing reagent, and xyloside having a chemical structure selected from Table 2 and/or Table 3.
TABLE 2

| 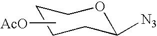 | 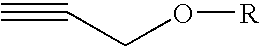 | 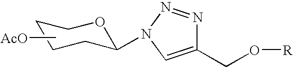 |
|---|---|---|
| 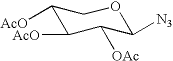 | 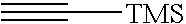 | 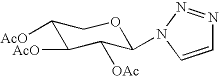 |
| | 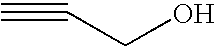 | 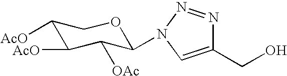 |
| | 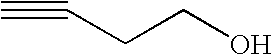 | 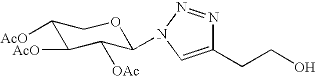 |
| | 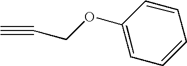 | 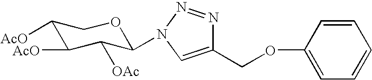 |
| | 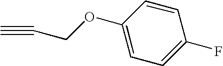 | 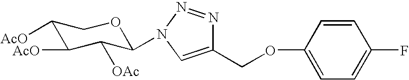 |
| | 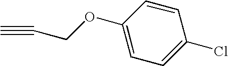 | 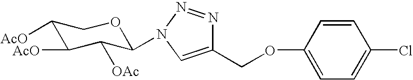 |
| | 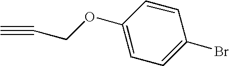 | 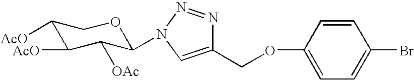 |
| | 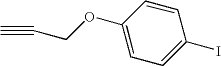 | 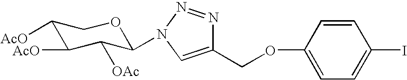 |
| | 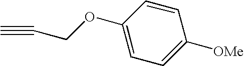 | 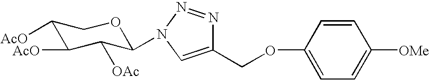 |
| | 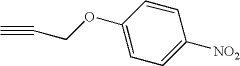 | 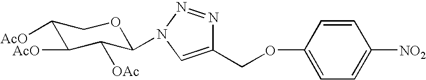 |
| | 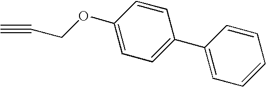 | 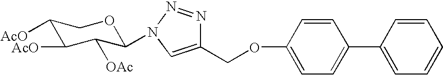 |
| | 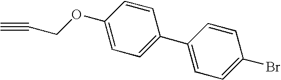 | 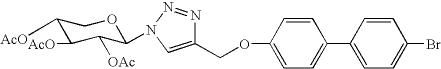 |
| | 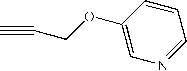 | 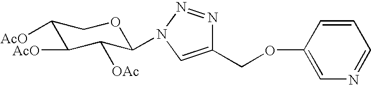 |
| | 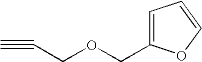 | 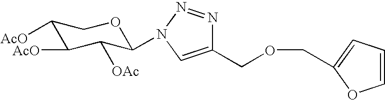 |
| | 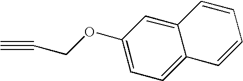 | 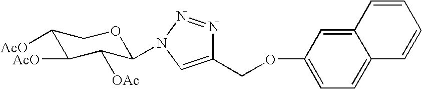 |
| | 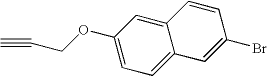 | 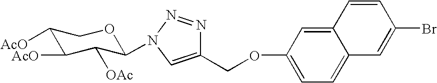 |
| | 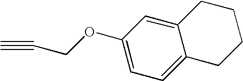 | 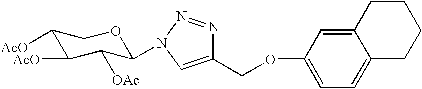 |
| | 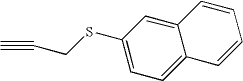 | 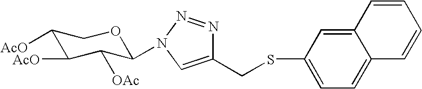 |
| | 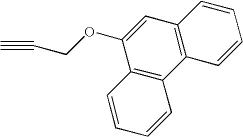 | 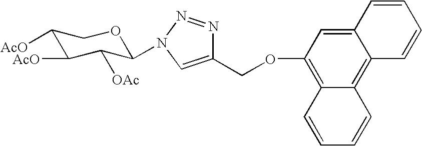 |
| | 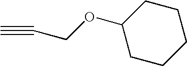 | 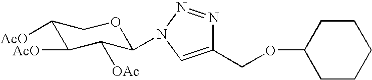 |

TABLE 3

| 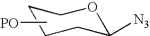 | 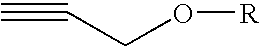 | 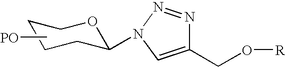 |
|---|---|---|
| 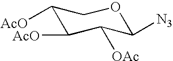 | 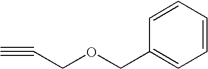 | 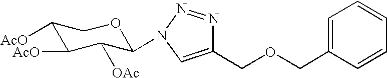 |
| | 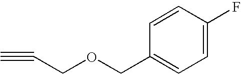 | 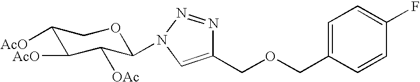 |
| | 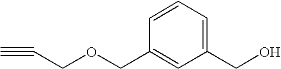 | 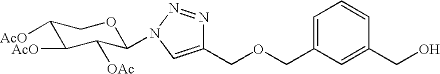 |
| | 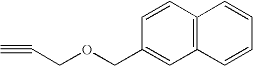 | 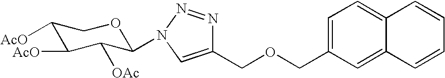 |
| | 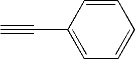 | 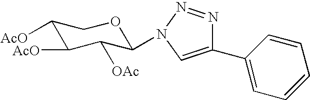 |
| | 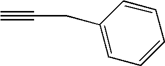 | 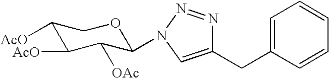 |
| 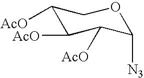 | 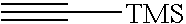 | 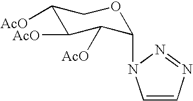 |
| | 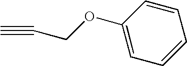 | 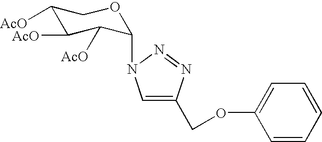 |
| | 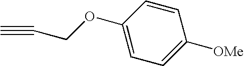 | 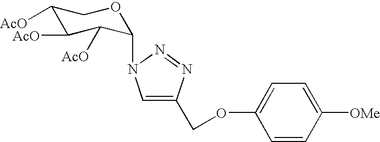 |
| | 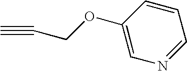 | 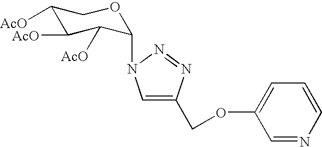 |
| | 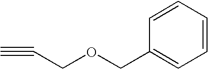 | 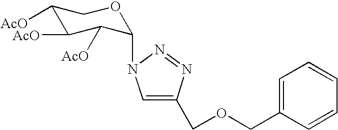 |
